(19) United States
(12) Patent Application Publication
(10) Pub. No.: US 2004/0152997 A1
(43) Pub. Date: Aug. 5, 2004

(54) Electrophysiological approaches to assess resection and tumor ablation margins and responses to drug therapy

(76) Inventor: Richard J. Davies, Saddle River, NJ (US)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/716,789

(22) Filed: Nov. 19, 2003

Related U.S. Application Data
(63) Continuation-in-part of application No. 10/151,233, filed on May 20, 2002.

(51) Int. Cl. A61B 5/05

(52) U.S. Cl. 600/547

(57) ABSTRACT

A method and system are provided for determining a condition of a selected region of tissue to facilitate the location of surgical resection margins. Electropotential and impedance are measured at one or more locations in an area of the body where tissue is to be removed. An agent may be introduced into the region of tissue to enhance electrophysiological characteristics of that tissue. The condition of the tissue is measured by electropotential and impedance profile. Differences in the profile are used to determine the borders between normal and abnormal tissue so as to facilitate what tissue to resect. Methods and apparatus are also disclosed to determine the efficacy of various therapies.

### CROSS-REFERENCE TO RELATED APPLICATIONS

This application is a continuation-in-part of U.S. patent application Ser. No. 10/151,233, filed May 20, 2002. The disclosures of the above mentioned application are hereby incorporated by reference herein. Applicant also incorporates the disclosures of the application entitled Method and System for Detecting Electrophysiological Changes in Pre-Cancerous and Cancerous Breast Tissue and Epithelium, Richard J. Davies, inventor, filed on this day of Nov. 19, 2003.

### BACKGROUND OF THE INVENTION

The present invention relates generally to the detection of abnormal or cancerous tissue and, more particularly, to the detection of changes in electrophysiological characteristics of abnormal or cancerous tissue related to the functional, structural, and topographic relationships of the tissue during the development of malignancy. These measurements may be made in the absence and/or presence of pharmacological or hormonal agents to reveal and accentuate electrophysiological characteristics indicative of abnormal or cancerous tissue.

Cancer is a leading cause of death in both men and women in the United States. Difficulty in detecting abnormal pre-cancerous or cancerous tissue before treatment options become non-viable is one reason for the high mortality rate. Detecting the presence of abnormal or cancerous tissues is difficult, in part, because such tissues are largely located deep within the body, thus requiring expensive, complex, invasive, and/or uncomfortable procedures. For this reason, the use of detection procedures is often restricted until a patient is experiencing symptoms related to the abnormal tissue. Many forms of cancers or tumors, however, require extended periods of time to attain a detectable size (and thus to produce significant symptoms or signs in the patient). It is often too late for effective treatment by the time the cancer or tumor is detected using currently available diagnostic modalities.

One proposed method for early detection of cancerous and pre-cancerous tissue includes measuring of the electrical impedance of biological tissue. For example, U.S. Pat. No. 3,949,736 discloses a low-level electric current passed through tissue, with a measurement of the voltage drop across the tissue providing an indirect indication of the overall tissue impedance. This method teaches that a change in impedance of the tissue is associated with an abnormal condition of the cells composing the tissue, indicating a tumor, carcinoma, or other abnormal biological condition. This disclosure, however, does not discuss either an increase or decrease in impedance associated with abnormal cells, nor does it specifically address tumor cells.

One disadvantage of this and similar systems is that the inherent DC electrical properties of the epithelium are not considered. Many common malignancies develop in an epithelium, often the cell layer that lines a hollow organ, such as the bowel, or in ductal structures, such as the breast or prostate. Epithelial tissue maintains a transepithelial electropotential (TEP) that may be altered by the malignancy process. Early in the malignant process, the epithelium may lose its transepithelial potential, particularly when compared to epithelium some distance away from the developing malignancy. Thus, the combination of transepithelial electropotential measurements with impedance may be more accurate in diagnosing pre-cancerous and cancerous conditions.

Another disadvantage of the above referenced system is that the frequency range is not defined. Certain information may be obtained about cells according to the range of frequencies selected. Different frequency bands may be associated with different structural or functional aspects of the tissue. See, for example, F. A. Duck, Physical Properties of Tissues, London: Academic Press, 2001; K. R. Foster, H. P. Schwan, Dielectric properties of tissues and biological materials: a critical review, Crit. Rev. Biomed. Eng., 1989, 17(1): 25-104. For example at high frequencies, such as >1 GHz, molecular structure has a dominating effect on the relaxation characteristics of the impedance profile. Relaxation characteristics include the delay in response of a tissue to a change in the applied electric field. For example, an applied AC current results in a voltage change across the tissue which will be delayed, or phase shifted, because of the impedance characteristics of the tissue. Relaxation and dispersion characteristics of tissue vary according to the frequency of the applied signal.

At lower frequencies, such as <100 Hz, or the so called α-dispersion range, alterations in ion transport and charge accumulations at large cell membrane interfaces dominate the relaxation characteristics of the impedance profile. In the frequency range between a few kHz and 1 MHz, or the so-called β-dispersion range, cell structure dominates the relaxation characteristics of the epithelial impedance profile. Within this range at low kHz frequencies, most of the applied current passes between the cells through the paracellular pathway and tight junctions. At higher frequencies in the β-dispersion range the current can penetrate the cell membrane and therefore passes both between and through the cells, and the current density will depend on the composition and volume of the cytoplasm and cell nucleus.

Characteristic alterations occur in the ion transport of an epithelium during the process of malignant transformation affecting the impedance characteristics of the epithelium measured at frequencies in the α-dispersion range. Later in the malignant process, structural alterations with opening of the tight junctions and decreasing resistance of the paracellular pathways, together with changes in the composition and volume of the cell cytoplasm and nucleus, affect the impedance measured in the β-dispersion range.

Another disadvantage of the above referenced system is that the topography of altered impedance is not examined. By spacing the measuring electrodes differently, the epithelium can be probed to different depths. The depth that is measured by two surface electrodes is approximately half the distance between the electrodes. Therefore, electrodes 1 mm apart will measure the impedance of the underlying epithelium to a depth of approximately 500 microns. It is known, for example, that the thickness of bowel epithelium increases at the edge of a developing tumor to 1356±208μ compared with 716±112μ in normal bowel. D. Kristt, et al. Patterns of proliferative changes in crypts bordering colonic tumors: zonal histology and cell cycle marker expression. Pathol. Oncol. Res 1999; 5(4): 297-303. By comparing the measured impedance between electrodes spaced approximately 2.8 mm apart with the impedance of electrodes spaced approximately 1.4 mm apart, information about the deeper and thickened epithelium may be obtained. See, for example, L. Emtestam & S. Ollmar. Electrical impedance index in human skin: measurements after occlusion, in 5 anatomical regions and in mild irritant contact dermatitis. Contact Dermatitis 1993; 28(2): 104-108.

Another disadvantage of the above referenced methods is that they do not probe the specific conductive pathways that are altered during the malignant process. For example, potassium conductance is reduced in the surface epithelium of the colon early in the malignant process.

Other patents, such as U.S. Pat. Nos. 4,955,383 and 5,099,844, disclose that surface electropotential measurements may be used to diagnose cancer. Empirical measurements, however, are difficult to interpret and use in diagnosis. For example, the above referenced inventions diagnose cancer by measuring voltage differences (differentials) between one region of the breast and another and then comparing them with measurements in the opposite breast. Changes in the measured surface potential may be related to differences in the impedance characteristics of the overlying skin. This fact is ignored by the above referenced and similar inventions, resulting in a diagnostic accuracy of 72% or less. J. Cuzick et al. Electropotential measurements as a new diagnostic modality for breast cancer. Lancet 1998; 352 (9125): 359-363; M. Faupel et al. Electropotential evaluation as a new technique for diagnosing breast lesions. Eur. J. Radiol. 1997; 24 (1): 33-38.

Other inventions that use AC measurement, such as U.S. Pat. No. 6,308,097, also have a lower accuracy than may be possible with a combination of DC potential measurements and AC impedance measurements. The above referenced system diagnoses cancer by only measuring decreased impedance (increased conductance) over a cancer.

Another potential source of information for the detection of abnormal tissue is the measurement of transport alterations in the mucosa. Epithelial cells line the surfaces of the body and act as a barrier to isolate the body from the outside world. Not only do epithelial cells serve to insulate the body, but they also modify the body's environment by transporting salts, nutrients, and water across the cell barrier while maintaining their own cytoplasmic environment within fairly narrow limits. One mechanism by which the epithelial layer withstands the constant battering is by continuous proliferation and replacement of the barrier. This continued cell proliferation may partly explain why more than 80% of cancers are of epithelial cell origin.

It is known that the addition of serum to quiescent fibroblasts results in rapid cell membrane depolarization. Cell membrane depolarization is an early event that may be associated with cell division. Depolarization induced by growth factors appears biphasic in some instances, but cell division may be stimulated without depolarization. Cell membrane depolarization is temporally associated with Na⁺ influx, and the influx persists after repolarization has occurred. Although the initial Na⁺ influx may result in depolarization, the increase in sodium transport may not cease once the cell membrane has been repolarized, possibly due to Na/K ATPase pump activation. Other studies also support that Na⁺ transport is altered during cell activation. In addition to altered Na⁺ transport, transport of K⁺ and of Cl⁻ is altered during cell proliferation.

A number of studies have demonstrated that proliferating cells are relatively depolarized when compared to those that are quiescent or non-dividing. Differentiation is associated with the expression of specific ion channels. Additional studies indicate that cell membrane depolarization occurs because of alterations in ionic fluxes, intracellular ionic composition, and transport mechanisms that are associated with cell proliferation.

Intracellular Ca²⁺ (Ca²⁺ᵢ) and intracellular pH (pHᵢ) are increased by mitogen activation. Cell proliferation may be initiated following the activation of phosphatidylinositol which releases two second messengers, 1,2-diacylglycerol and inosotol-1,4,5-triphosphate, which trigger Ca²⁺ᵢ release from internal stores. Ca²⁺ᵢ and pHᵢ may then alter the gating of various ion channels in the cell membrane, which are responsible for maintaining the voltage of the cell membrane. Therefore, there is the potential for interaction between other intracellular messengers, ion transport mechanisms, and cell membrane potential. Most studies have been performed in transformed and cultured cells and not in intact epithelia during the development of cancer, so that it is largely unknown how up-regulated proliferation affects cell membrane potential, transepithelial potential, epithelial impedance, and ion transport during carcinogenesis.

It was known that cancer cells are relatively depolarized compared to non-transformed cells. It has been suggested that sustained cell membrane depolarization results in continuous cellular proliferation and that malignant transformation results as a consequence of sustained depolarization and a failure of the cell to repolarize after cell division. C. D. Cone Jr., Unified theory on the basic mechanism of normal mitotic control and oncogenesis. J. Theor. Biol. 1971; 30(1): 151-181; C. D. Cone Jr., C. M. Cone. Induction of mitosis in mature neurons in central nervous system by sustained depolarization. Science 1976; 192 (4235): 155-158; C. D. Cone, Jr. The role of the surface electrical transmembrane potential in normal and malignant mitogenesis. Ann. N.Y. Acad. Sci. 1974; 238: 420-435. A number of studies have demonstrated that cell membrane depolarization occurs during transformation and carcinogenesis. Other studies have demonstrated that a single ras-mutation may result in altered ion transport and cell membrane depolarization. Y. Huang, S. G. Rane, Single channel study of a Ca(2+)-activated K+ current associated with ras induced cell transformation. J. Physiol. 1993; 461: 601-618. For example, there is a progressive depolarization of the colonocyte cell membrane during 1,2 dimethylhydrazine (DMH)-induced colon cancer in CF₁ mice. The V_{A} (apical membrane voltage) measured with intracellular microelectrodes in histologically “normal” colonic epithelium depolarized from −74.9 mV to −61.4 mV after 6 weeks of DMH treatment and to −34 mV by 20 weeks of treatment.

While epithelial cells normally maintain their intracellular sodium concentration within a narrow range, electronmicroprobe analysis shows that cancer cells exhibit cytoplasmic sodium/potassium ratios that are three to five times greater than those found in their non-transformed ones. These observations partly explain the electrical depolarization observed in malignant or pre-malignant tissues, because of the loss of K⁺ or Na⁺ gradients across the cell membrane.

In addition to cell membrane depolarization, and altered intracellular ionic activity, other studies have shown that there may be a decrease in electrogenic sodium transport and activation of non-electrogenic transporters during the development of epithelial malignancies. These changes may occur as a consequence of altered intracellular ionic composition. Other specific ion transport alterations have been described in colon, prostate, breast, uterine cervix, melanoma, urothelium, and pancreas during proliferation, differentiation, apoptosis, and carcinogenesis.

Apoptosis or physiological cell death is down-regulated during the development of malignancy. Ion transport mechanisms affected by apoptosis include the influx of Ca²⁺, non-selective Ca²⁺-permeable cation channels, calcium-activated chloride channels, and K⁺-Cl⁻-cotransport. J. A. Kim et al. Involvement of Ca2+ influx in the mechanism of tamoxifen-induced apoptosis in Hep2G human hepatoblastoma cells. Cancer Lett. 1999; 147(1-2): 115-123; A. A. Gutierrez et al. Activation of a Ca2+-permeable cation channel by two different inducers of apoptosis in a human prostatic cancer cell line. J. Physiol. 1999; 517 (Pt. 1): 95-107; J. V. Tapia-Vieyra, J. Mas-Oliva. Apoptosis and cell death channels in prostate cancer. Arch. Med. Res. 2001; 32(3): 175-185; R. C. Elble, B. U. Pauli. Tumor Suppression by a Proapoptotic Calcium-Activated Chloride Channel in Mammary Epithelium. J. Biol. Chem. 2001; 276(44): 40510-40517.

Loss of cell-to-cell communication occurs during carcinogenesis. This results in defective electrical coupling between cells, which is mediated via ions and small molecules through gap junctions, which in turn influences the electrical properties of epithelia.

Epithelial cells are bound together by tight junctions, which consist of cell-to-cell adhesion molecules. These adhesion proteins regulate the paracellular transport of molecules and ions between cells and are dynamic structures that can tighten the epithelium, preventing the movement of substances, or loosen allowing substances to pass between cells. Tight junctions consist of integral membrane proteins, claudins, occludins and JAMs (junctional adhesion molecules). Tight junctions will open and close in response to intra and extracellular stimuli.

A number of substances will open or close tight junctions. The proinflammatory agent TGF-alpha, cytokines, IGF and VEGF opens tight junctions. Zonula occludens toxin, nitric oxide donors, and phorbol esters also reversibly open tight junctions. Other substances close tight junctions including calcium, H2 antagonists and retinoids. Various hormones such as prolactin and glucocorticoids will also regulate the tight junctions. Other substances added as drug formulations act as non-specific tight junction modulators including chitosan and wheat germ agglutinin.

The above referenced substances and others may act directly or indirectly on the tight junction proteins, which are altered during carcinogenesis. For example claudin-7 is lost in breast ductal epithelium during the development of breast cancer. The response of the tight junctions varies according to the malignant state of the epithelium and their constituent proteins. As a result the opening or closing of tight junctions is affected by the malignant state of the epithelium.

Polyps or overtly malignant lesions may develop in a background of disordered proliferation and altered transepithelial ion transport. Experimental animal studies of large bowel cancer have demonstrated that transepithelial depolarization is an early feature of the pre-malignant state. In nasal polyp studies, the lesions had a higher transepithelial potential, but these lesions were not pre-malignant in the same sense as an adenomatous or pre-malignant colonic polyp, that are usually depolarized. Electrical depolarization has been found in biopsies of malignant breast tissue. Recently alterations in impedance have been found to be associated with the pre-malignant or cancerous state in breast and bowel.

DC electrical potential alterations have been reported to be useful to diagnose non-malignant conditions such as cystic fibrosis, cancer in animal models, human cells or isolated tissue, and in man. Differences in impedance between normal tissue and cancer have been described in animal models in vitro and have been applied to in vivo cancer diagnosis. DC potential measurements have not been combined with impedance measurements to diagnose cancer, however, because electrophysiological alterations that accompany the development of cancer are generally not fully characterized. Transepithelial depolarization is an early event during carcinogenesis, which may affect a significant region of the epithelium (a “field defect”). This depolarization is accompanied by functional changes in the epithelium including ion transport and impedance alterations. Early on in the process these take the form of increased impedance because of decreased specific electrogenic ion transport processes. As the tumor begins to develop in the pre-malignant epithelium, structural changes occur in the transformed cells such as a breakdown in tight junctions and nuclear atypia. The structural changes result in a marked reduction in the impedance of the tumor. The pattern and gradient of electrical changes in the epithelium permit the diagnosis of cancer from a combination of DC electrical and impedance measurements. Another reason that DC electropotential and impedance measurements have not been successfully applied to cancer diagnosis is that transepithelial potential and impedance may be quite variable and are affected by the hydration state, dietary salt intake, diurnal or cyclical variation in hormonal level, or non-specific inflammatory changes and other factors. In the absence of knowledge about the physiological variables which influence transepithelial potential and impedance these kinds of measurements may not be reliable to diagnose pre-malignancy or cancer. Furthermore a detailed understanding of the functional and morphological alterations that occur during carcinogenesis permits appropriate electrical probing for a specifically identified ion transport change that is altered during cancer development. For example knowledge that electrogenic sodium absorption is reduced during cancer development in the colon permits the use of sodium channel blockers (e.g., amiloride) or varying sodium concentration in the ECM to examine whether there is an inhibitable component of sodium conductance. By varying the depth of the measurement (by measuring the voltage drop across differently space electrodes), it is possible to obtain topographic and depth information about the cancerous changes in the epithelium.

The diagnostic accuracy of current technology using DC electropotentials or impedance alone has significant limitations. Sensitivity and specificity for DC electrical measurements in the breast have been reported as 90% and 55% respectively and 93% and 65% for impedance measurements. This would result in an overall diagnostic accuracy of between 72-79%, which is probably too low to result in widespread adoption. J. Cuzick et al. Electropotential measurements as a new diagnostic modality for breast cancer. Lancet 1998; 352 (9125): 359-363; A. Malich et al. Electrical impedance scanning for classifying suspicious breast lesions: first results. Eur. Radiol. 2000; 10(10): 1555-1561. The combination of DC electrical potentials and impedance spectroscopy may result in a diagnostic accuracy of greater than 90% which will lead to improved clinical utility.

Thus, there remains a need for effective, practical methods of detecting abnormal tissue.

### SUMMARY OF THE INVENTION

To overcome problems and inadequacies associated with prior methods, abnormal or cancerous tissue is characterized using DC measurements and impedance measurements in combination. DC measurements provide information about the functional state of the epithelium and can detect early pre-malignant changes and an adjacent malignancy. Impedance measurements at different frequencies using differently spaced electrodes provide depth and topographic information to give both structural (high frequency range) and functional (low frequency range) information about the tissue being probed. Abnormal or cancerous tissue can be detected and characterized by detecting and measuring transport alterations in mucosal tissues, using ionic substitutions and/or pharmacological and hormonal manipulations to determine the presence of abnormal pre-cancerous or cancerous cells. A baseline level of transepithelial DC potential, impedance, or other electrophysiological property that is sensitive to alterations in transport in epithelia is measured in the tissue to be evaluated. An agent may be introduced to enhance the transport or make it possible to detect the transport alteration. The transepithelial DC potential and/or impedance of the tissue (or other electrophysiological property that may reflect or make it possible to detect alterations in the transport) are then measured. Based on the agent introduced and the measured electrophysiological parameter, the condition of the tissue is determined.

A method and system are provided for determining a condition of a selected region of epithelial tissue. At least two current-passing electrodes are located in proximity to or in contact with a first surface of the selected region of the tissue. Alternatively, the current passing electrodes may pass current across the tissue or epithelium. For example, current may be passed between the urethra and surface of the prostate, accessed per rectum; between the abdominal wall and the bowel mucosal surface; between the skin surface of the breast and the central breast ducts accessed by central duct catheter or ductoscope. A plurality of measuring electrodes is located in contact with or in proximity with the first surface of the selected region of tissue as well. A signal is established between the current-passing electrodes. One or more of the measuring electrodes measures impedance associated with the established signal. Alternatively a three electrode system may be used for measurements whereby one electrode is used for both current injection and voltage recording. An agent is introduced into the region of tissue. The condition of the tissue is determined based on the effect of the agent on measured DC transepithelial potential impedance or other electrophysiological characteristics. The electrodes in the described methods and apparatus can be used in contact with, in proximity to, over, or inserted into the tissue being examined. It should be understood that where the method is described in an embodiment as encompassing one of these arrangements, it is contemplated that it can also be used interchangeably with the other. For example, where the method is described as having an electrode in contact with the tissue, the method can also be used with the electrode inserted into or in proximity to the tissue. Similarly, where the method is described as having an electrode in proximity to the tissue, it is contemplated that the electrode can also be in contact with or inserted into the tissue.

In order to more accurately detect transport alterations in abnormal pre-cancerous or cancerous epithelial tissue, a pharmacological agent may be introduced to manipulate the tissue. Pharmacological agents may include agonists of specific ion transport and electrical activity, antagonists of specific ion transport and electrical activity, ionic substitutions, and/or hormonal or growth factor stimulation or inhibition of electrical activity.

Depending on the location of the tissue to be investigated, a number of methods may be used to administer the pharmacological or hormonal agents. One exemplary method includes introducing the agent directly to the tissue being investigated, via either direct contact or injection. Another exemplary method includes applying the agent to the skin surface, wherein the agent acts transcutaneously, or through the skin. Yet another exemplary method includes electroporation, wherein the epithelium or surface is made permeable by the passage of alternating current via electrodes in contact or penetrating the organ or epithelium of interest. The agent then passive diffuses into the organ and its constituent cells. Additional exemplary methods include via inhalation, oral administration, lavage, gavage, enema, parenteral injection into a vein or artery, sublingually or via the buccal mucosa, or via intraperitoneal administration. One skilled in the art will appreciate that other methods are possible and that the method chosen is determined by the tissue to be investigated.

Thus, systems and methods consistent with the present invention use a combination of transepithelial electropotential and impedance measurements to diagnose pre-malignancy or cancer. Further, systems and methods consistent with the present invention use a defined set of frequencies in combination to characterize functional and structural alterations in pre-malignancy and cancer. By using spaced electrodes the present invention may provide topographic and geometrical (depth) information about the epithelium under examination to diagnose pre-malignancy and cancer. In one embodiment, systems and methods of the present invention use electrodes with specially formulated ECMs to provide functional information about the epithelium to diagnose pre-malignancy and cancer.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate one embodiment of the invention and together with the description, serve to explain the principles of the invention. In the Figures:

FIG. 1 is a schematic diagram of a DC and AC impedance measuring device, consistent with an embodiment of the present invention;

FIG. 2 illustrates an exemplary embodiment of a device suitable for use with systems and methods consistent with the present invention;

FIG. 3 illustrates another exemplary embodiment of a device suitable for use with systems and methods consistent with the present invention;

FIGS. 4A and 4B illustrates other exemplary embodiments of a device suitable for use with systems and methods consistent with the present invention;

FIGS. 5A and 5B illustrate the short circuit current associated with human colonic epithelium ex-vivo;

FIG. 6 is a photomicrograph illustrating electrophysiologic and histologic alterations that may be present in colonic cancer;

FIG. 7 illustrates measurements of epithelial electropotential in a patient with rectal cancer;

FIG. 8 illustrates varying ionic content and the effect on transepithelial conductance in human breast epithelium;

FIG. 9 illustrates measurements of cell membrane potential in human breast epithelial cells;

FIG. 10 illustrates the effect of increasing estradiol on the transepithelial potential in benign and malignant breast epithelia;

FIG. 11 illustrates conductance and electropotential measurements made over the surface of the breast in women with and without breast cancer;

FIG. 12 illustrates the measurement of electropotential at the surface of the breast, and variation of the measurement during menstrual cycle;

FIG. 13 illustrates measurements of cell membrane potential in human prostatic epithelial cell under different growth conditions;

FIG. 14 illustrates measurements of electropotential in a patient with normal prostate; and

FIG. 15 illustrates measurements of electropotential in a patient with prostate cancer.

### DETAILED DESCRIPTION

Reference will now be made in detail to an embodiment of the invention, an example of which is illustrated in the accompanying drawings. Wherever possible, the same reference numbers will be used throughout the drawings to refer to the same or like parts.

In order to combine DC transepithelial measurement with impedance measurements, it may be necessary to obtain baseline measurement of the DC potential using the voltage sensing electrodes, referenced to a low impedance surface electrode, or the blood stream via an IV, or the interstitial body fluid via a needle electrode or electrode that permeabilizes the overlying epidermis or other epithelium, or other body reference point. The electrodes may contain different ionic concentrations, pharmacological agents, or hormones in their ECMs. As used in this description, an ECM is a medium that permits transmission of electrical signals between the surface being measured and the electrode. An agent includes any ionic concentration, pharmacological agent, hormone, or other compound added to the ECM or otherwise introduced to the tissue under investigation, selected to provide further information about the condition of the tissue. In another embodiment the concentrations of agents may be changed using a flow through system.

Electroconductive media can include conductive fluids, creams or gels used with external or internal electrodes to reduce the impedance (resistance to alternating current) of the contact between the electrode surface and the skin or epithelial surface. In the case DC electrodes it is also desirable that the ECM results in the lowest DC offset at the electrode surface, or an offset that can be measured. The ECM will often contain a hydrogel that will draw fluid and electrolytes from deeper layers of the skin to establish electrical contact with the surface electrode. Electrodes that are used to pass current require ECMs with high conductance. Usually this is accomplished by using ECMs with high electrolyte content. The electrolytes frequently used are KCl (potassium chloride) because of the similar ionic mobility of these two ions in free solution, so that electrode polarization is less of a problem than when ions of different mobility are used. Other ions such as sodium may be used in ECM formulations, and the higher electrolyte concentration result in more rapid electrode equilibration.

In situations where estimations will be made of the permeability of the epithelium to specific ions, the concentration of K in the ECM will be varied so that the conductance of the epithelium to potassium may be measured electrophysiologically. An enhancer or permeant may be added to the ECM to increase the conductance of the underlying skin to the electrolyte in the ECM. Other approaches include mild surface abrasion to reduce surface skin resistance or silicon electrodes, which just penetrate the stratum corneum to reduce skin surface resistance.

In order to measure the depth of the impedance alteration, a voltage drop is made between electrodes with different spacing. Spacing is determined by knowledge of the depth to be probed. Similarly, two different frequency ranges will be used to measure functional and structural changes at different depths.

In order to more accurately detect the functional transport alterations at different depths in abnormal pre-cancerous or cancerous epithelial tissue, an agent, such as a pharmacological agent, may introduced to manipulate the tissue, while electrically probing the tissue at different frequencies and monitoring the voltage drop between differently spaced electrodes. Pharmacological agents include agonists of specific ion transport and electrical activity, antagonists of specific ion transport and electrical activity, ionic substitutions, and/or hormonal or growth factor stimulation, which modulates, inhibits or stimulates electrical activity.

Depending on the location of the tissue to be investigated, a number of methods may be used to administer the pharmacological or hormonal agents. One exemplary method includes introducing the agent directly to the tissue being investigated, via either direct contact or injection. Another exemplary method includes applying the agent to the skin surface, wherein the agent acts transcutaneously, or through the skin. Another exemplary method includes electroporation, wherein the epithelium or surface is made permeable by the passage of alternating current via electrodes in contact with or penetrating the organ or epithelium of interest. The agent then passively diffuses into the organ and its constituent cells. Additional exemplary methods include via inhalation, oral administration, lavage, gavage, enema, parenteral injection into a vein or artery, sublingually or via the buccal mucosa, or via intraperitoneal administration. One skilled in the art will appreciate that other methods are possible and that the method chosen is determined by the tissue to be investigated.

Based on the agent introduced and the tissue being investigated, measurements of electrophysiological properties, such as impedance, are performed. Other properties that can be measured includes, transepithelial potential, changes in spontaneous oscillations in transepithelial potential or impedance associated with the malignant state, and time delay in a propagation signal between electrodes, which indicates a change or loss of gap-junction function. The results of these measurements are then used to determine the condition of the investigated tissue. For example, research has indicated that specific ion transport processes are altered during the development of cancer. For example, a loss of electrogenic Na⁺ transport, an up-regulation in Na/H exchange, a down-regulation in K⁺ conductance, a decrease in basal Cl-absorption, and a down-regulation in c-AMP (cyclic adenosine-3′,5′-cyclic monophosphate) stimulated Cl⁻ secretion have been observed.

Thus, by administering agents appropriate to the particular epithelial tissue and measuring the associated electrophysiological characteristics, it is possible to detect abnormal pre-cancerous or cancerous tissue while the development of such tissue is at an early stage. The method and system of the present invention is applicable to any epithelial derived cancer, such as, but not limited to, prostate, colon, breast, esophageal, and nasopharyngeal cancers, as well as other epithelial malignancies, such as lung, gastric, uterine cervix, endometrial, skin, and bladder.

Specifically, in cancers affecting mucosal or epithelial tissues, transport alterations may be sufficiently large to suggest that they are a consequence of an early mutation, affecting a large number of cells (i.e., a field defect). In this case, they may be exploited as potential biomarkers for determining which patients should be either more frequently monitored, or conversely, may be used to identify particular regions of mucosa that require biopsy. The latter is especially helpful in the case of flat adenomas or dysplasia, which are more difficult to detect physically than, for example, polyps.

A number of variations are possible for devices to be used with the present invention. Further, within a device design, there are a number of aspects that may be varied. These variations, and others, are described below.

One probe or other device includes a plurality of miniaturized electrodes in recessed wells. Disposable commercially available silicon chips processing, such as filtering, may perform surface recording and initial electronic processing. Each ECM solution or agent may be specific to the individual electrode and reservoir on the chip. Thus, for one measurement, a particular set of electrodes is used. For another measurement, for example, at a different ionic concentration, a different set of electrodes is used. While this produces some variations, as the electrodes for one measurement are not located at the same points as for another, this system provides generally reliable results.

An alternative approach is to use fewer electrodes and use a flow-through or microfluidic system to change solutions and agents. Specifically, solutions or agents are changed by passing small amounts of electrical current to move solution or agent through channels and out through pores in the surface of the probe. In this embodiment, the electrode remains in contact with the same region of the epithelium, thus eliminating region-to-region variation in measurement. This approach requires time for equilibration between different solutions.

In detecting the presence of abnormal pre-cancerous or cancerous breast tissue, a hand-held probe is provided for obtaining surface measurements at the skin. The probe may include electrodes for passing current as well as for measuring. An impedance measurement may be taken between the nipple cup electrode and the hand-held probe, or may be taken between electrodes on the hand-held probe. After taking initial DC measurements, a wetting/permeabilizing agent may be introduced to reduce skin impedance. The agent may be introduced using a microfluidic approach, as described above, to move fluid to the surface of the electrodes. Alternatively, surface electrodes that just penetrate the stratum corneum may be used to decrease impedance.

Fluids for use with the present inventions could include various electrolyte solutions such as physiologic saline (e.g. Ringers) with or without pharmacological agents. One preferable electrolyte solution to infuse into the ductal system will represent a physiological Ringer solution. Typically this consists of NaCl 6 g, KCl 0.075 g, CaCl₂ 0.1 g, NaHCO₃ 0.1 g, and smaller concentrations of sodium hyper and hypophosphate at a physiological pH of 7.4. Other electrolyte solution may be used were the electrolyte comprises approximately 1% of the volume of the solute. Hypertonic or hypotonic solutions that are greater or less than 1% may be used in provocative testing of the epithelium and/or tumor. The concentration of Na, K and Cl will be adjusted under different conditions to evaluate the conductance and permeability of the epithelium. Different pharmacological agents such as amiloride (to block electrogenic sodium absorption), Forskolin (or similar drugs to raise cyclic-AMP) and hormones such as prolactin or estradiol can also be infused with the Ringer solution to examine the electrophysiological response of the epithelium and tumor to these agents. Similarly, the calcium concentration of the infusate will be varied to alter the tight junction permeability and measure the electrophysiological response of the epithelium to this manipulation. Dexamethasone may be infused to decrease the permeability of the tight junctions, and the electrophysiological response will be measured.

Although specific examples have been given of drugs and hormones that may be used in “challenge” testing of the epithelium and tumor, any agonist or antagonist of specific ionic transport, or tight-junctional integrity, known to be affected during carcinogenesis may be used, particularly when it is known to influence the electrophysiological properties of the epithelium or tumor.

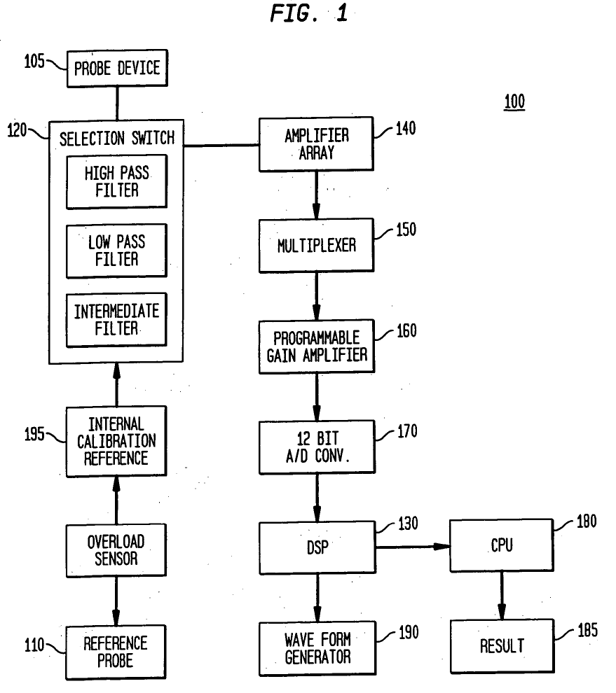

Regardless, of the configuration of the device, FIG. 1 is a schematic of a DC and AC impedance measurement system 100 used in cancer diagnosis, consistent with the present invention. The system 100 interfaces with a probe device 105 including multiple electrodes, wherein the actual implementation of the probe device 105 depends on the organ and condition under test. The probe device 105 may incorporate the electrodes attached to a glove, needle, body cavity, endoscopic, or surface probe. A reference probe 110 may take the form of an intravenous probe, skin surface probe, or epithelial surface reference probe depending on the test situation and organ under investigation.

To avoid stray capacitances, the electrodes may be connected via shielded wires to a selection switch 120 which may select a specific probe 105 following a command from the Digital Signal Processor (DSP) 130. The selection switch 120 also selects the appropriate filter interfaced to the probe 105, such that a low pass filter is used during DC measurements and/or an intermediate or high pass filter is used during the AC impedance measurements. The selection switch 120 passes the current to an amplifier array 140 which may be comprised of multiple amplifiers or switch the signals from different electrodes through the same amplifiers when multiple electrodes are employed. In a preferred embodiment digital or analogue lock-in amplifiers are used to detect minute signals buried in noise. This enables the measurement of the signal of interest as an amplitude modulation on a reference frequency. The switching element may average, sample, or select the signal of interest depending on the context of the measurement. This processing of the signal will be controlled by the DSP following commands from the CPU. The signals then pass to a multiplexer 150, and are serialized before conversion from an analogue to a digital signal by the ADC. A programmable gain amplifier 160 matches the input signal to the range of the ADC 170. The output of the ADC 170 passes to the DSP 130. The DSP 130 processes the information to calculate the DC potential and its pattern on the epithelial or skin surface as well as over the region of suspicion. In addition the impedance at varying depth and response of the DC potential and impedance to different ECM concentrations of ions, drug, hormones, or other agent are used to estimate the probability of cancer. The results are then sent to the CPU 180 to give a test result 185.

Alternatively the signal interpretation may partly or completely take place in the CPU 180. An arbitrary waveform generator 190 or sine wave frequency generator will be used to send a composite waveform signal to the probe electrodes and tissue under test. The measured signal response (in the case of the composite wave form stimulus) may be deconvolved using FFT (Fast Fourier Transforms) in the DSP 130 or CPU 180 from which the impedance profile is measured under the different test conditions. An internal calibration reference 195 is used for internal calibration of the system for impedance measurements. DC calibration may be performed externally, calibrating the probe being utilized against an external reference electrolyte solution.

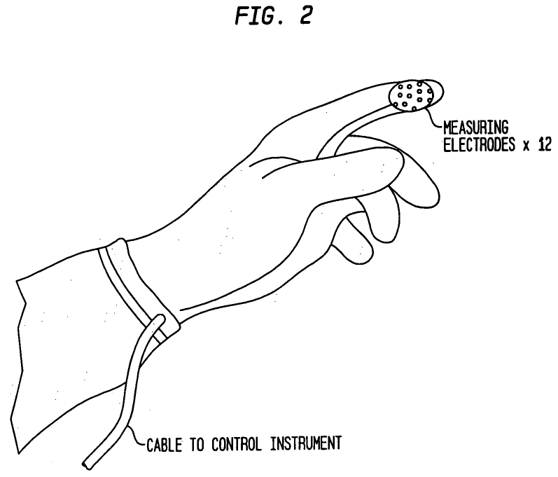

FIG. 2 illustrates a glove that may be used, for example, in diagnosis of prostate cancer or as a screening test for colorectal neoplasia. Multiple sensor electrode arrays may be attached to an examining glove together with current passing electrodes. The individual electrodes may be recessed and ECMs with different composition may be used to pharmacologically, electrophysiologically, or hormonally probe the epithelium under test. Spacing of the electrodes may be greater for the prostate configuration than for other organ systems so that deeper tissue may be electrically probed and the impedance of the deeper tissue evaluated. The electrodes will be interfaced via electrical wire, or wireless technology, with the device described in FIG. 1 above.

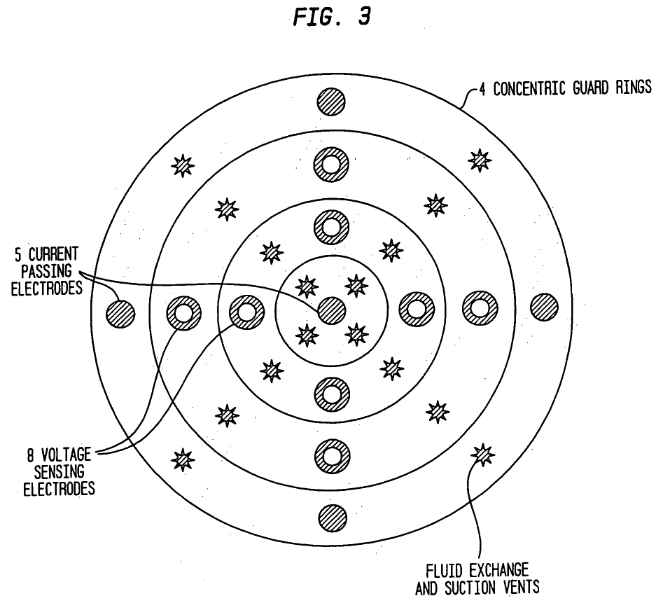

FIG. 3 is a schematic of an endoscopic probe, consistent with the present invention, which may be placed in contact with the epithelium endoscopically. This probe may either be placed passively in contact with the epithelium or held in place by pneumatic suction over the region of interest. Ports are in place for the exchange of solutions or for fluid exchange and suction. Guard rings may be incorporated to prevent cross-talk between electrodes and to force current from the contact surface into the epithelium. In this configuration there are four current passing electrodes each positioned radially 90° apart. This permits current to be passed and the voltage response to be measured in perpendicular fields. This enables the effects of surface asymmetry on impedance (such as occurs with aberrant crypt foci) to be measured. Electrodes may be slightly recessed so as not to influence current density measured at the surface.

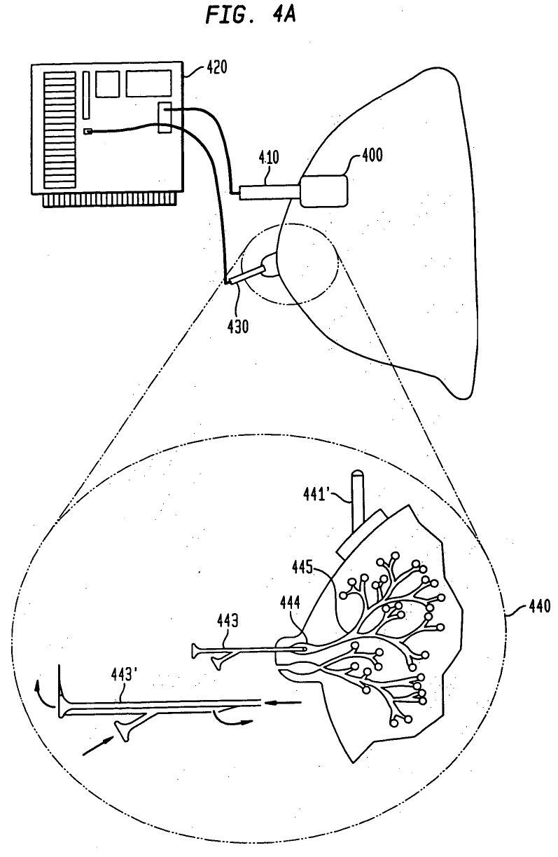

FIG. 4A includes a handheld probe 400, consistent with the present invention, which may be applied to the surface of the breast. The probe may include a handle 410. The probe 400 may be attached, either directly or indirectly using, for example, wireless technology, to a measurement device 420. The probe 400 may be referenced to an intravenous electrode, a skin surface electrode, or other ground. In one embodiment, illustrated in FIG. 4A, the reference is a nipple electrode or ductal probe 430, illustrated in greater detail at close-up 440. One advantage of this configuration is that DC electropotential and impedance can be measured between the nipple electrode 430 and the probe 400. The measurement is thus a combination of the DC potentials and impedance of the breast ductal epithelium, non-ductal breast parenchyma, and the skin.

Referring to close-up 440, the ductal probe is inserted into one of several ductal orifices that open onto the surface of the nipple. Ductal probe 443 is shown within a ductal sinus 444, which drains a larger collecting duct 445.

Another advantage of using a nipple electrode is that a solution for irrigating the ductal system may be exchanged through the probe, permitting introduction of pharmacological and/or hormonal agents. As shown in magnified nipple probe 443, 443′ fluid can be exchanged through a side port. Fluid may be infused into the duct and aspirated at the proximal end (away from the nipple) of the nipple probe. Different electrolyte solutions may be infused into the duct to measure altered permeability of the ductal epithelium to specific ions or the epithelium may be probed with different drugs to identify regions of abnormality. Estradiol, or other hormonal agents, may be infused into a breast duct to measure the abnormal electrical response associated with pre-malignant or malignant changes in the epithelium.

It should be understood that different configurations may also be used, such as a modified Sartorius cup that applies suction to the nipple. With this configuration, gentle suction is applied to a cup placed over the nipple. Small amounts of fluid within the large ducts and duct sinues make contact with the electrolyte solution within the Sartorius cup, establishing electrical contact with the fluid filling the breast ducts. DC or AC measurements may then be made between the cup and a surface breast probe.

FIG. 4B illustrates the probe 400 of FIG. 4A in greater detail. The skin contact of the surface 450 is placed in contact with the breast. The surface electrodes 451 measure DC or AC voltages. The current passing electrodes 452 are used for impedance measurements. Probe 400 may also include one or more recessed wells containing one or more ECMs.

Further embodiments of this technique may involve the use of spaced electrodes to probe different depths of the breast, and the use of hormones, drugs, and other agents to differentially alter the impedance and transepithelial potential from benign and malignant breast tissue, measured at the skin surface. This enables further improvements in diagnostic accuracy.

### EXAMPLE 1

### Colon Cancer

In colon cancer, the following electrophysiological changes have been observed during the development of the abnormal tissue: loss of electrogenic Na+ transport, up-regulation in Na/H exchange, down-regulation in K⁺ conductance, decrease in basal Cl⁻ absorption, and down-regulation in c-AMP (cyclic adenosine-3′,5′-cyclic monophosphate) stimulated Cl⁻ secretion. A number of pharmacological and hormonal manipulations can be performed to detect these ion transport alterations.

By using electrolyte conductive medium (ECM) of different concentrations, the conductance of specific ions can be estimated and the response to different pharmacological probes can be determined. Different pharmacological agents are administered that influence electrophysiological properties of normal bowel, but have minimal or different effects on pre-cancerous or cancerous tissue. For example, glucocorticoids or mineralocorticoids, administered by injection or orally, increase the transepithelial electropotential (TEP) of normal colon, but have a lesser effect on pre-cancerous or cancerous tissue. These steroids up-regulate electrogenic sodium absorption, thereby decreasing sodium specific impedance in normal colon.

The measured TEP decreases in response to a topically applied amiloride (a sodium channel blocker) in normal colonic mucosa. This response is reduced by approximately 50% in pre-cancerous mucosa or by greater than 75% in cancerous mucosa. In addition, the loss in sodium conductance results in an increase of impedance of the surface epithelium. This ion transport alteration may be measured by determining the change in TEP as well as the basal impedance. In abnormal pre-cancerous or cancerous tissue, the TEP is lower, the response of the TEP to amiloride is less, and the increase in impedance (observed in normal colon in response to amiloride) is less in abnormal pre-cancerous or cancerous tissue. Similar pharmacological agents may be introduced that alter the effect of chloride or potassium ion transport, which affect abnormal pre-cancerous or cancerous tissue in a different manner than in normal colon tissue.

It is important to note that the impedance is higher, or conductance is generally lower, around the edge of the tumor or in the immediately adjacent pre-malignant epithelium. At more than 5-10 cm from the tumor the TEP is lower and ion specific impedances may be higher. In the tumor itself the impedance is lower (conductance higher). Measurement may be made over a suspected tumor, but also adjacent and some distance away from the suspected tumor to more accurately identify the cancerous or pre-cancerous tissue. There are also pharmacological differences between normal pre-cancerous and cancer tissue. Direct comparison between these different regions can used to make a more accurate diagnosis of cancer or premalignancy.

In one embodiment, electrophysiological measurements are performed using a series of two or more electrodes attached to an examining glove or probe. Some factors influencing the spacing of the electrode and the signal used include the depth of penetration desired and permeabilization of the surface epithelium using penetrating agents. A probe that permits variable frequency signals and varying electrode placement provides the most versatile arrangement, but a probe or glove providing a single frequency signal and/or static electrode placement may also be used.

Sodium: Sodium conductance and absorptive properties in the surface cells of the colonic epithelium are markedly attenuated in some pre-cancerous and cancerous cells. By measuring the impedance of the colonic epithelium using low frequency sine waves and closely placed electrodes, it is possible to determine the electrophysiological activity of the surface cells. Passive electrodes, placed between current-passing electrodes, measure the impedance, while ECMs of different sodium concentration may be used to reveal alterations of the specific ionic permeabilities of the epithelium. By using higher frequency sine waves and widely spaced electrodes and ECMs of varying sodium concentration, it is possible to estimate overall and ion-specific conductances of the deeper epithelium. A ratio may be determined, expressed as the change in surface to deep sodium conductance. The surface/deep sodium conductance ratio progressively decreases as tissue develops from at-risk, to pre-cancerous to cancerous tissue. The surface cells that are conductive to sodium are replaced by cells from the deeper epithelium that do not have as high a conductance. Therefore, the ratio of surface Na⁺ conductance/deep Na⁺ conductance goes from >2.0 to <1.0. Both the ratio and absolute number change. Measuring the ratio effectively normalizes the measurement for the particular individual and epithelial region under test.

A number of ECMs and pharmacological agents may be employed to characterize the sodium transport characteristics of colonic tissue. In one exemplary method, initial measurements are made using an electrolyte solution containing 10 mM KCl in the ECM, either in gel or solution, which interfaces between the electrode and the bowel wall. Measurements are taken relative to an intravenous reference electrode or a low impedance skin electrode, having a minimal offset voltage relative to the underlying extracellular fluid and bloodstream. The TEP is then measured at increasing levels of sodium, both in the absence and presence of amiloride or similar agent, such as benzamil, (10 μM-1 mM) to block electrogenic sodium transport. The difference between the two measurements will be the TEP attributable to the electrogenic sodium transport across the bowel epithelium. The electrogenic component of sodium transport is diminished by 40-50% in colonic epithelium that is at-risk or pre-cancerous.

One method for varying the sodium and/or pharmacological content during measurement include using one or more wells or reservoirs associated with each electrode, containing different concentrations of electrolyte and/or agent, so that the solution is not actually changed during measurement but the measurement occurs under different conditions with different electrodes and ECMs. Another method involves a flow-through solution change system, whereby solution changes may be automated while using fewer electrodes.

Potassium: Measurements similar to that described above, with reference to sodium, are performed with reference to potassium. Specifically, an early decrease in potassium conductance is associated with at-risk or pre-cancerous colonic epithelium. As cancer develops, potassium conductance becomes up regulated and potassium secretion may be enhanced. The decrease, and then increase, in potassium conductance enables not only identification of abnormal tissue, but also the determination of the condition of the tissue, as either normal, at-risk, pre-cancerous, or cancerous.

Impedance measurements may be performed at varying concentrations of potassium, using signals of varying frequency, and using variably spaced electrodes, thus providing an impedance profile including the superficial and deep epithelium. For example, one method of determining an impedance profile, with reference to potassium, is as follows: A TEP measurement is made using increasing concentrations of K⁺ and all measurements are performed using ECM containing amiloride or another blocker of the electrogenic Na⁺ pump to remove the contribution of electrogenic Na⁺ transport to TEP. Using the well method described above, the ECM in each well contains a combination of amiloride, bethanacol, forskolin, and 3-isobutyl-1-methylxanthine (IBMX). Each of the four wells contains varying K⁺ concentrations (between 10 and 80 mM), while maintaining the concentration of Na and Cl ions. These agents (bethanacol, forkskolin, and IBMX) depolarize the cell membrane by maximally opening Cl⁻ conduction channels in the surface cells of the colon. This cell membrane depolarization results in the opening of voltage-sensitive K+ channels in the cell membrane. Specifically, bethanacol (or carbacol) raises intracellular Ca²⁺ which opens Ca²⁺ sensitive K⁺ channels, as well as increasing chloride secretion opening up Cl⁻ channels. Other muscarinic agonists may produce similar results. Forkskolin increases adenyl cyclase, thereby raising intracellular c-AMP opening up K⁺-channels. IBMX, a phosphodiesterase inhibitor, may be used to raise c-AMP. Other agents, such as theophylline, may also be used to raise c-AMP. Agents, such as dibutyrl c-AMP, may be used to increase c-AMP directly. These agents maximally increase potassium conductance and secretion, permitting the identification of reduced potassium secretion and conductance associated with at-risk or pre-cancerous tissue.

Another such method employs measurements with a series of varying KCl concentrations in contact with the colonic mucosa, such as 10, 20, 40, and 80 mM KCl. Electrodes containing 10 μM-1 mM amiloride in the ECM are used to measure TEP and impedance, both in the presence and absence of K⁺-channel blockers, such as 20 mM TEA (tetraethyl ammonium) and 5 mM barium. The TEP is lower than normal in the at-risk and pre-cancerous tissue. The impedance is lower than normal in the cancerous tissue. In transitional tissue or tissue adjacent to developing cancer, impedance may be higher than normal.

Chloride: Similar to the methods for sodium and potassium described above, chloride conductance can be used to determine abnormal pre-cancerous and cancerous tissue. Chloride conductance occurs mainly at the base of the crypt (or deep) in normal epithelium. In cancerous tissue, the epithelial cells closer to the surface of the crypt become more conductive to chloride, albeit at a lower level of conductance than observed in the base. The ratio of chloride conductance between the surface and the base, as estimated from impedance measurements, can be used to characterize colonic tissue as either normal, at-risk, pre-cancerous, or cancerous. Specifically, at-risk and pre-cancerous epithelium exhibits an overall decrease in chloride conductance, with an increase in the surface/base ratio. As the tissue progresses to cancerous, the overall chloride conductance increases and is accompanied by increased Cl⁻ secretion. The surface/base ratio may become less discriminatory, however, because normal epithelial morphology is lost in a malignant tumor.

As with potassium, chloride-dependent TEP is measured using increasing concentrations of Cl⁻. Measurements are made in the presence of an ECM containing a sodium pump blocker agent, such as amiloride, in order to negate the contribution of electrogenic Na⁺ transport, and agents, such as bethanacol, forskolin, and IBMX to maximally open Cl⁻ conduction channels in the surface cells of the colon. The wells have Cl-concentrations varying between 15 and 120 mM, while maintaining the concentrations of Na and K ions and keeping osmolality constant. In at-risk and pre-cancerous tissue, the Cl⁻ is reduced. Additionally, the TEP is lower than normal. In cancerous tissue, the basal Cl⁻ secretion and Cl⁻ conductance is increased.

Drug Provocation: In addition to the ionic manipulations described above, the colon responds to a number of different hormones, growth factors, and diets by changing the ion transport characteristics of the epithelium. For example, aldosterone (a mineralocorticoid) and dexamethasone (a glucocorticoid) both increase electrogenic sodium absorption and potassium secretion in the colon. In normal colon, sodium conductance is increased in surface cells and the epithelium hyperpolarizes, or becomes more negative in the lumen. Potassium conductance increases in the deeper cells. In at-risk, pre-cancerous, or cancerous tissue, however, this response is significantly different. The hyperpolarization and increase in sodium conductance is markedly diminished. The increase in the potassium conductance in the basal cells of the crypt is much less than occurs in normal colon. Thus, agents and treatments that affect the ion transport characteristics of the epithelium may be used to enhance differences between normal and abnormal colon tissue in impedance measurements and/or other measurements of the electrical characteristics. A high-potassium, low-sodium diet will produce similar effects in a normal bowel. Other agents may be administered directly to the surface of the bowel and produce similar effects in normal epithelium. Carbenoxolone, for example, when administered rectally, increases TEP in normal bowel, but has a lesser effect on pre-cancerous or cancerous tissue. It causes an increase in TEP because it inactivates 11β-HSD (11-beta hydroxysteroid dehydrogenase). Cortisol has mineralocorticoid effects on the bowel and increases electrogenic sodium absorption and therefore increases TEP in normal but not in abnormal or cancerous bowel epithelium.

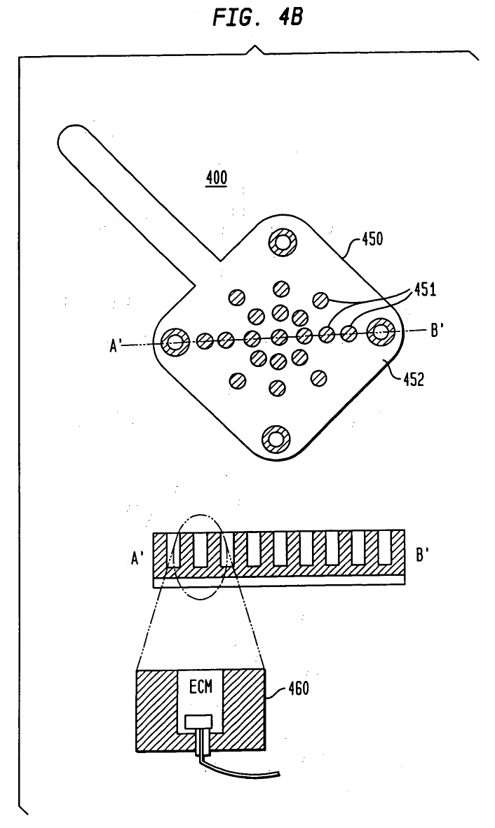

FIG. 5A demonstrates the short circuit current of human colonic epithelium ex-vivo. The figure demonstrates the time course along the x-axis while varying the potassium gradient across the tissue. The potassium permeability of the apical membrane of human colonic mucosa (P^{K}ₐ) was determined in surgical specimens of controls and grossly normal-appearing mucosa obtained 10-30 cm proximal to colorectal adenocarcinomas. The mucosa was mounted in Ussing chambers and the basolateral membrane resistance and voltage were nullified by elevating the K⁺ in the serosal bathing solution. The apical sodium (Na⁺) conductance was blocked with 0.1 mM amiloride. This protocol reduces the equivalent circuit model of the epithelium to an apical membrane conductance and electromotive force in parallel with the paracellular pathway as has been verified by microelectrode studies. Increasing serosal K⁺ caused the I_{sc} to become negative (−140 μA/cm²) in normal colon after which 30 mM mucosal TEA caused an abrupt increase in Isc corresponding to block of apical K⁺ channels. In cancer-bearing colon the reduction in Isc is to −65 μA/cm². The serosal bath was remained constant at 125 mM [K].

FIG. 5B demonstrates that ΔI_{sc}, determined with respect to the I_{sc} at 125 mM mucosal K, is a linear function of the concentration gradient, Δ[K]. Because the voltage across the apical membrane is zero under these conditions and the paracellular pathway is nonselective, the P^{K}ₐ (apical potassium permeability) can be calculated using the Fick equation—i.e., I_{sc}=F×P^{K}ₐΔ[K] where F is the Faraday constant and Δ[K] is the concentration difference for K⁺ across the epithelium. FIG. 5b demonstrates mean±sem values for I_{sc} in both normal and premalignant human distal colon. The apical K⁺ permeability of controls was 9.34×10⁻⁶ cm/sec and this was significantly reduced by 50% in premalignant human mucosa to 4.45×10⁻⁶ cm/sec. P^{K}ₐ could also be calculated for the change in I_{sc} when the K⁺ channels were blocked with TEA, assuming complete block. This resulted somewhat lower values of 6.4×10⁻⁶ cm/sec and 3.8×10⁻⁶ cm/sec corresponding to a 40% reduction in p^{K}ₐ.

These observations show that there is a field change in the K⁺ permeability and conductance of human colon, during the development of cancer. Impedance measurements, DC measurement using electrodes with different potassium gradients together with specific drugs, such as amiloride to block the contributions of electrogenic Na⁺ transport to the electrical properties of the bowel are useful to diagnose colon cancer.

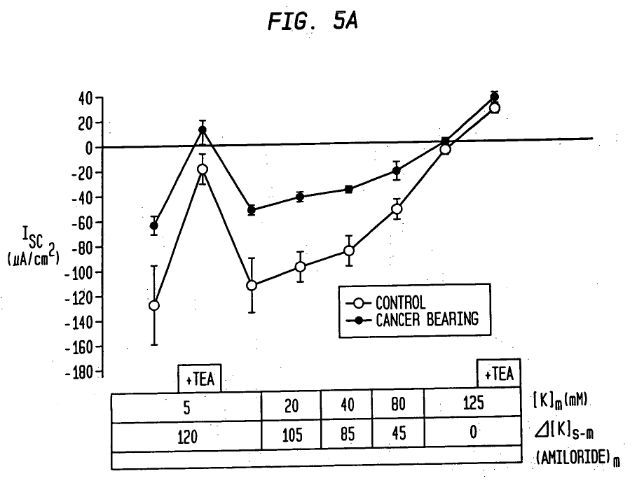

FIG. 6 is a photomicrograph which illustrates some of the complexities associated with electrophysiological and histological alterations that occur in the development of colonic cancer. The cancer is a 10 mm in diameter, invasive and an ulcerated lesion that could easily be missed at colonoscopy (because it is a depressed lesion). The cancer is depolarized to 0 mV with a much higher conductance than the surrounding epithelium. The surrounding or adjacent epithelium is also depolarized at about −20 mV but has a higher impedance than the cancer or normal epithelium. Note that the darker layer, the epithelium (e), is on the top surface. This is one cell layer thick, but form crypts, like inverted test tubes with proliferation and secretory function at the base and differentiated cells and absorptive function at the mouth. The inferior layer (m) is the muscle layer of the bowel. This small tumor has already invaded the muscle layer. More distant epithelium is also depolarized but to a lesser degree at −40 mV. Potassium conductance is decreased in this morphologically normal-appearing epithelium. Chloride secretion is also decreased compared to the tumor, which may actively secrete chloride. The sodium conductance, G_{Na}, is decreased and the Na/H exchanger is upregulated. The colonic mucosa tends to be thickened with elongated crypts in the region of the developing cancer (adjacent zone). Most of the impedance resides in the epithelial layer, and therefore a higher impedance below 750 μm indicates an epithelial thickening associated with cancer. Recognizing the electrophysiological pattern enables a diagnosis of cancer to be made, i.e. an electrophysiological virtual biopsy.

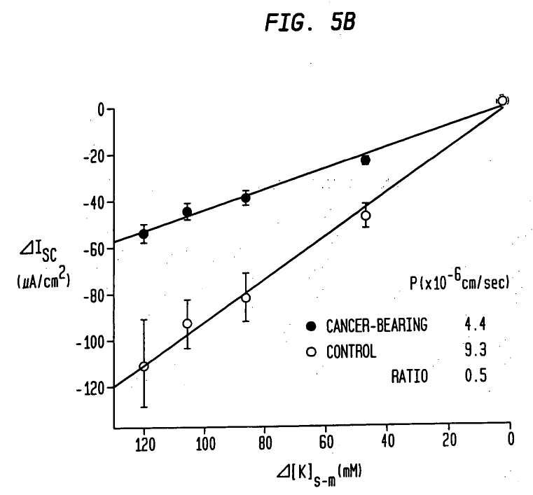

FIG. 7 demonstrates measurements of surface mucosal (epithelial) electropotential referenced to the serosal surface on a freshly excised specimen of pelvic colon and rectum from a 45-year-old male with an ulcerated rectal carcinoma. Following resection the specimen was immediately opened in a longitudinal direction and surface electropotential measurements were made using different ECMs. Following excision there is usually a decrease in the electropotential (“run-down”) of 5-10 mV in the first 5-10 minutes, although the relative electropotential differences from region to region remain similar.

The “starburst” at the lower end of the figure, 2-3 cms from the anal canal and 5 cms from the anal verge has an electropotential of +10 mV measured over the surface of the tumor (left hand column “Normosol Ringers's”). Normosol Ringer's is a physiological saline solution containing approximately 5 mM K⁺. The normal mucosal surface electropotential is −50 to −70 mV in the rectum. As measurement are taken some distance from the tumor the bowel remains depolarized even up to 20 cm from the edge of the tumor where readings of −40 to −45 mV are observed. This region is depolarized relative to normal colon where levels of less than −50 mV are observed.

When electropotential measurements are made in normal colon using an ECM with a higher K⁺ concentration an increase in electropotential (increased positivity) of 20 mV or greater is frequently observed. This is because the normal colon is selectively permeable to K⁺ and the increased ECM K⁺ concentration sets up a diffusion potential for K⁺ across the ion-selective conductance pathways. In the cancer bearing colon K+ conductance decreases in the region of the developing tumor as well as some distance from it (“field-cancerization”). Up to 5 cm from the developing cancer there is essentially no change in the measured electropotential when the ECM is changed from 5 to 30 mM K⁺ (change from left column (“Normosol Ringer's”) to middle column “30 mM KCl” in figure). Up to 20 cm from the tumor the change in electropotential does not exceed 15 mV (−45 to −30 mv) 20 cms from the edge of the tumor. A further increase in the K⁺ concentration of the ECM results in small increases in positivity away from the tumor or an anomalous decrease in positivity near or at the tumor, suggesting that a diffusion gradient for a different ion (other than K⁺) is set-up in the vicinity of the tumor.

Depolarization in combination with altered K⁺ conductance and permeability may be used to diagnose the presence of cancer or increased risk of cancer. Altered K⁺ conductance is observed before tumors develop in the bowel. Combination with simultaneous impedance measurements increases diagnostic accuracy.

### EXAMPLE 2

### Breast Cancer

As mentioned above, impedance and DC electrical potential have been used separately at the skin's surface to diagnose breast cancer. In the current invention, the impedance characteristics of the overlying skin or epithelium are measured and factored in to the diagnostic interpretation of the data. For example the surface potential may be more positive (or less negative) than the reference site because of increased conductance of the overlying skin, rather than because of an underlying tumor.

The electrodes are placed over the suspicious region and the passive DC potential is measured. Then AC impedance measurements are made as discussed below. The variable impedance properties of the overlying skin may attenuate or increase the measured DC surface electropotentials. Alternatively, impedance measurements at different frequencies may initially include a superimposed continuous sine wave on top of an applied DC voltage. Phase, DC voltage and AC voltage will be measured. The resistance of the skin or other epithelium at AC and a different resistance at DC are measured. Under DC conditions since there is no phase shift we are able to measure the transepithelial potential at the surface. The capacitive properties of the skin allow the underlying breast epithelial and tumor potential to be measured at the skin surface.

Once the ECM results in “wetting” of the skin surface there is pseudo-exponential decay in the skin surface potential using the above referenced approach. Ions in the ECM diffuse through the skin and make it more conductive, particularly because of changes in the skin parallel resistance. The time constant for this decay is inversely proportional to the concentration and ionic strength of the gel. Once the skin is rendered more conductive by the ECM the capacitive coupling of the surface to the underlying potential of the tumor or the surrounding epithelium is lost so that the measured potential now reflects an offset and diffusion potential at the electrode-ECM-skin interfaces.

The use of pharmacological and/or hormonal agents, however, in combination with both impedance and DC electrical potential, provides an even more effective method for detecting abnormal pre-cancerous or cancerous breast tissue. Breast cancer develops within a background of disordered proliferation, which primarily affects the terminal ductal lobular units (TDLUs). The TDLUs are lined by epithelial cells, which maintain a TEP. In regions of up-regulated proliferation, the ducts are depolarized. The depolarization of ducts under the skin surface is capacitively coupled with the overlying skin, which results in skin depolarization. When a tumor develops in a region of up-regulated proliferation the overlying breast skin becomes further depolarized compared with other regions of the breast and the impedance of the cancerous breast tissue decreases. Electrophysiological responses in TEP and impedance change under the influence of hormones and menstrual cycle.

For example, the electrophysiological response of breast tissue to 17-β-estradiol has been observed to be different in pre-cancerous or cancerous tissue than in normal breast tissue. In one method of present invention, estradiol is introduced directly into the duct or systematically following sublingual administration of 17-β-estradiol (4 mg). This agent produces a rapid response, which peaks at approximately 20 minutes. The electrophysiological response depends, in part, on the stage of the patient's menstrual cycle, as well as the condition of the breast tissue. Specifically, in normal breast tissue, a rise in TEP will occur during the follicular (or early) phase. In pre-cancerous or cancerous tissue, this response is abrogated. Post-menopausal women at risk for breast cancer may have an exaggerated TEP response to estradiol because of up-regulated estrogen receptors on epithelial cell surfaces.

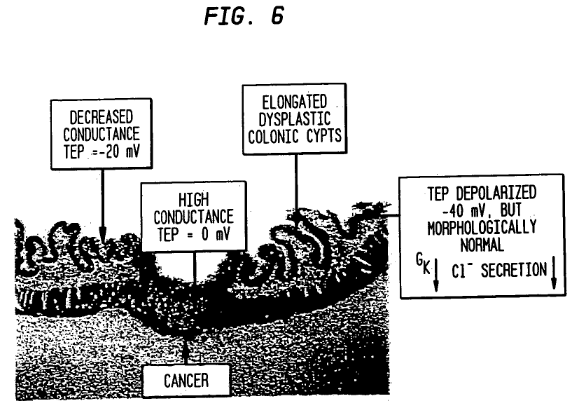

FIG. 8 demonstrates the effect of varying the ionic content of the bathing Ringers solution on transepithelial conductance. The human breast epithelial cells were grown as monolayers on Millipore filters and grew to confluence in 7 to 10 days. The epithelia were then mounted in modified Ussing chambers and the DC conductances were measured using a voltage clamp. The conductance was measured by passing a 2 μA current pulse for 200 milliseconds and measuring the DC voltage response and calculating the transepithelial conductance (y-axis), and plotting it against time (x-axis). The conductance was measured first in standard Ringer solution, then in a sodium-free Ringer, then returned to standard Ringer, then in a potassium-free Ringer and finally returning to standard Ringer solution while maintaining normal osmolality during the studies.

The upper plot (filled squares and solid line) demonstrates the conductance of benign human breast epithelia grown as a monolayer. The conductance is higher in the benign epithelial cells. The Na⁺ and K⁺ components of conductance are approximately, 10 and 5 mS.cm² respectively.

The lower plot (filled circles and dotted line) demonstrates the conductance of malignant human breast epithelia grown as a monolayer. The conductance is significantly lower in the malignant epithelial cells. The Na⁺ and K⁺ components of conductance are approximately, 4 and 1 mS.cm⁻² respectively.

In malignant tumors as opposed to monolayers of malignant epithelial cells the tight junction between cells break down and the tumor becomes more conductive than either benign or malignant epithelial monolayers. This observation may be exploited in the diagnosis of breast cancer. The lower conductance of the epithelium around a developing tumor, together with a region of high conductance at the site of the malignancy, may be used to more accurately diagnose breast cancer. Using electrodes with ECMs with different ionic composition will permit the specific ionic conductances to be used in cancer diagnosis. For example a high conductance region with a surrounding area of low K-conductance is indicative of breast cancer, A high conductance area with a surrounding region of normal conductance may be more indicative of fibrocystic disease (a benign process).

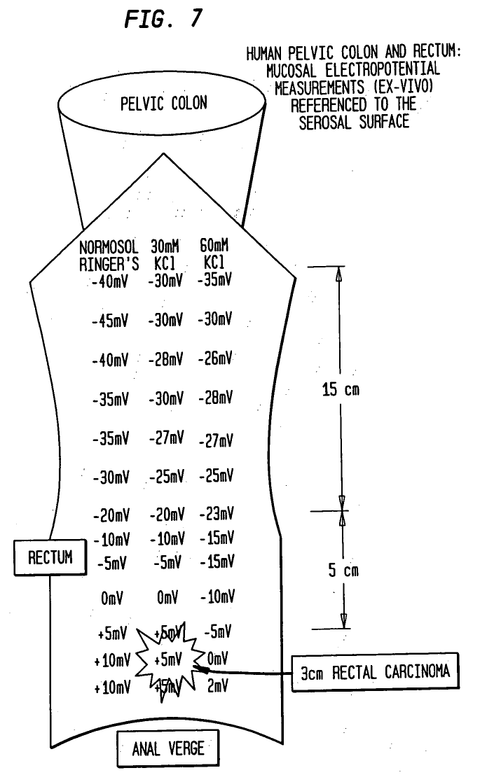

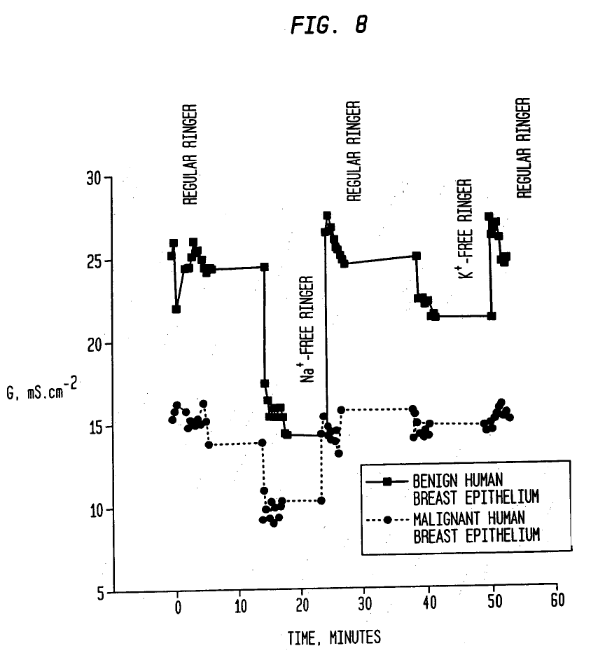

FIG. 9 demonstrates measurements of cell membrane potential (Ψ) in human breast epithelial cells. Measurements were made using a potentiometric fluorescent probe, and ratiometric measurements, which are calibrated using valinomycin and K⁺-gradients. Ψs were measured in the presence (closed circles) and absence (open circles) of estradiol (the active metabolite of estrogen). Each symbol is the mean measurement. The upper error bar is the standard error of the mean, and the lower error bar is the 95% confidence level for the observations. The addition of estrogen to cultured breast epithelial cells results in an instantaneous increase in Ψ (data not shown) as well as transepithelial potential see FIG. 10. Transepithelial potential (V_{T}) of an epithelium is the sum of the apical (luminal) cell membrane potential (V_{A}) and the basolateral (abluminal) cell membrane potential (V_{BL}). Therefore V_{T}=V_{A}+V_{BL}(changes in V_{A} and V_{BL} will therefore alter V_{T} or transepithelial potential).

FIG. 9 demonstrates that benign breast epithelial cells have a Ψ of approximately −50 mV in the absence of estradiol and −70 mV when estradiol is added to the culture media. Malignant and transformed cells have a Ψ of between −31 and −35 mV in the absence of estradiol and approximately 50 mV when estradiol is present in the culture medium.

The difference in the electrical properties may be exploited to diagnose breast cancer in vivo. Surface electropotential measurements are a combination of the transepithelial potential, tumor potential and overlying skin potential. Physiological doses of estradiol may be administered to the patient to increase Ψ and the sustained effect of estradiol results in an increase in transepithelial potential and tumor potential measured as an increase in surface electropotential. The increase following sustained exposure (as opposed to the instantaneous response) is less in malignant than benign breast tissue.

It should be noted that the instantaneous response, illustrated in FIG. 10, is greater in malignant epithelia, whereas the chronic or sustained exposure to estradiol results in a lower increase in TEP (transepithelial electropotential) in malignant cells. Concurrent measurement of surface electropotential and impedance allow the more accurate diagnosis of cancer. FIG. 10 demonstrates the instantaneous effect of increasing doses of estradiol on the transepithelial potential (TEP) of benign and malignant human breast epithelial cells. The cells were grown as monolayers on Millipore filters and grew to confluence in 7 to 10 days. The epithelia were then mounted in modified Ussing chambers and the TEP was measured using a voltage clamp. Increasing doses of estradiol between 0 and 0.8 μM were added (x-axis). The transepithelial potential was measured after each addition and the TEP was measured (y-axis).

The different dose response is apparent for benign and malignant epithelia. Malignant epithelia have a lower TEP but undergo an instantaneous increase in TEP of approximately 9 mV (becomes more electronegative and reaches a level of <6 mV) after exposure to only 0.1 μM estradiol and then depolarize to approximately −2 mV with increasing doses of estradiol up to about 0.5 μM. Benign epithelia have a lesser response to increasing doses of estradiol and do not peak until almost 0.3 μM and then remain persistently elevated (higher electro negativity), unlike the malignant epithelia, with increasing doses of estradiol.

This difference in dose response may be exploited to diagnose breast cancer. Estradiol, or other estrogens, at a low dose will be administered systemically, transcutaneously, or by other route. The instantaneous response of the surface electropotential and impedance may then be used to diagnose breast cancer with improved accuracy over existing diagnostic modalities using impedance or DC measurement alone.

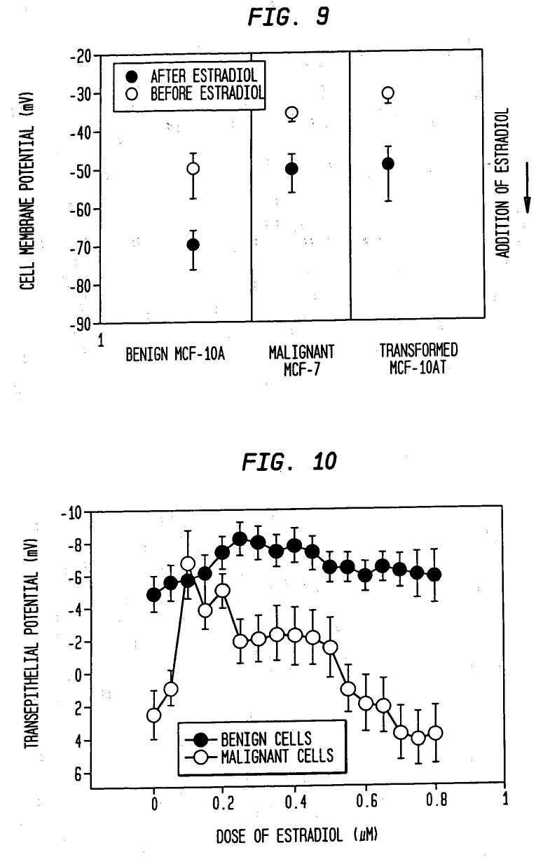

FIG. 11 shows conductance measurements made at 2000 Hz at the surface of the breast. At this frequency the influence of the overlying skin impedance is less. There is still however some variable component of skin impedance, which results in significant variability of the measurement as evidenced by the overlapping error bars. Each symbol represents the median measurement with error bars the standard deviation of the mean.

Open symbols represent measurements made in patients with a biopsy proven malignancy, while closed symbols represent measurements made in patients whose subsequent biopsy proved to be a benign process such as fibrocystic disease. Malignant lesions are often associated with surrounding breast epithelium that demonstrates up-regulated proliferation. These regions (“adjacent region”) are depolarized and may have a lower conductance than either over the region of malignancy. This decreased conductance may be because of decreased K+-conductance of the adjacent and pre-malignant epithelium as I have observed in human colon.

Each of the three groups of symbols represents measurements from over a suspicious lesion or region, then the adjacent region, and then over normal breast in an uninvolved quadrant of the breast. The first two symbols (circles) in each of the three groups are impedance measurements where the median value is plotted against the left y-axis as conductance in mS.cm². The second two symbols (squares) is the surface electrical potential measured in mV and plotted against the right y-axis; each division equals 5 mV. The third two symbols (triangles) is the electrical index for benign and malignant lesions and is in arbitrary units and is derived from the conductance and surface potential measurement. It is immediately apparent that there is less overlap in the error bars (standard deviation of the mean). Therefore breast cancer can be more accurately diagnosed using a combination of surface potential measurement and AC-impedance measurements. Further enhancements of this technique will involve the use of spaced electrodes to probe different depths of the breast, and the use of the hormones, drugs and other agents to differentially alter the impedance and transepithelial potential from benign and malignant breast tissue, and measured at the skin surface. This will enable further improvements in diagnostic accuracy.

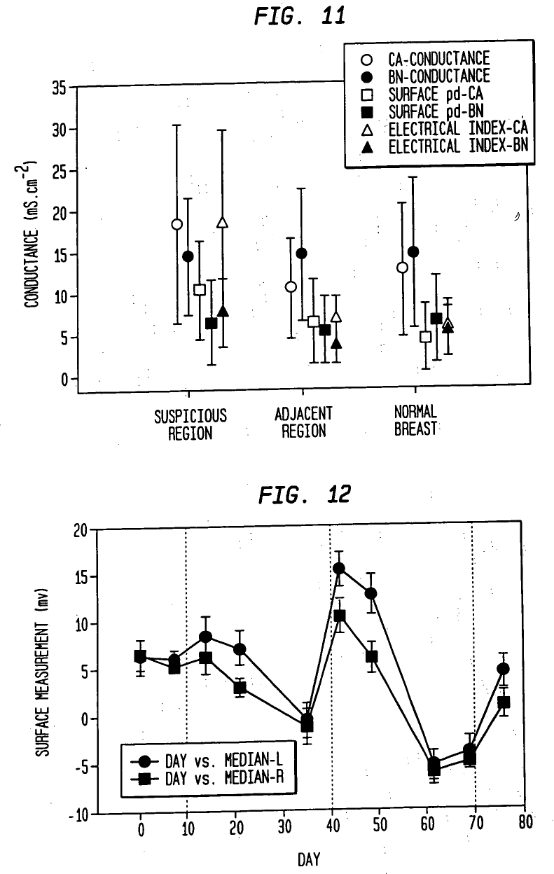

It should be understood that the surface potential measurement of breast tissue varies based on the position of the woman in her menstrual cycle. FIG. 12 illustrates this variance. This figure demonstrates electropotential measurements taken over the surface of each breast at 8 different locations with an array of 8 electrodes on each breast referenced to an electrode on the skin of the upper abdomen. Measurements are taken with error bars equal to the standard error of the mean. Filled circles and filled squares represent the median value from the left and right breast respectively. The vertical dotted line is the first day of each menstrual cycle.

It can be seen that the median values for each breast tend to track one another with lower values in the first half of menstrual cycle (follicular phase) and higher values in the latter part of cycle (luteal phase). Although the measured electrical values are not completely superimposed, because of other factors affecting the electropotential of the breast, it can be seen that the lowest levels of electropotential are observed 8-10 days before menstruation and the rise to the highest levels around the time of menstruation. This may be because estradiol levels are higher in the second part of menstrual cycle and directly affect breast surface electropotential.

The cyclical pattern of electropotential activity when a breast cancer or proliferative lesion is present is quite different. Similarly higher levels of surface electropotential are observed when measurements were made in the afternoon compared with the morning. This information can be exploited in a number of different ways. Measurement of the surface potential and impedance at different times during cycle enables a more accurate diagnosis because of a different cyclical change in surface electropotential (i.e., the peak to peak change in potential is less over a malignant region, relative to normal areas of the breast). Secondly, estradiol or another agent that changes the electropotential of the breast may be administered systemically, topically (transdermal), or by other means, and the drug or hormone-induced change in surface potential may be used as a provocative test to diagnose breast cancer.

In one embodiment of the present invention, breast cancer may be diagnosed by examining the basal conductance state of the paracellular pathway of the epithelium. For example, in the breast, a substance known to affect the conductance of the tight junctions may be infused into the duct, or administered by other mean, and the transepithelial impedance and/or the DC potential of the breast is measured, before and after the administration of the agent, using a combination of surface, nipple, ductal or other electrodes. The difference in the transepithelial electrical response of the tight junctions to the agent in normal compared to pre-malignant or malignant breast epithelium is then is used to diagnose the presence or absence of malignancy.

In these ways breast cancer can be more accurately diagnosed using a combination of surface potential measurement and AC-impedance measurements.

### EXAMPLE 3

### Nasopharyngeal Cancer

Using methods similar to those described with respect to colon cancer, it is possible to use pharmacological and hormonal agents to enhance electrophysiological alterations caused by nasopharyngeal cancer. One exemplary method would be a nasopharyngeal probe that would include wells providing for varying concentrations of K+ and would perform simple DC measurements.

### EXAMPLE 4

### Prostate

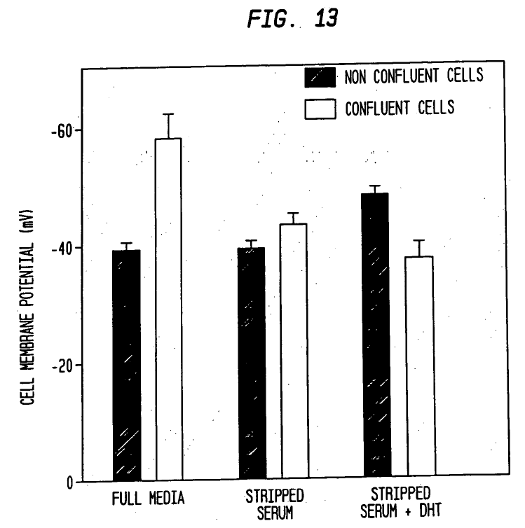

FIG. 13 represent measurements of cell membrane potential (ψ) in human prostatic epithelial cells under different growth conditions. A Voltage-sensitive FRET (fluorescent energy transfer) probe was used for potentiometric ratio measurements. It has two fluorescent components: CC₂-DMPE (Coumarin) and DISBAC₂(3) (Oxonol). The oxonol distributes itself on opposite sides of the cell membrane in a Nernstian manner according to the ψ. The voltage sensitive distribution of oxonol is transduced through a ratiometric fluorescence signal via the coumarin which is bound to the outside surface of the cell membrane thereby amplifying the fluorescence. Measurements were made using a fluorescence microscope and a digital imaging system. The ratio measurements are calibrated using Gramicidin D to depolarize the cell membrane and then varying the external K+-concentration. The calibrated cell membrane potential in mV is depicted on the y-axis.

The filled bars indicates the ψ of exponentially growing prostatic epithelial cells before they reach confluence, whereas the open bars depict the ψ of the cells once they reach confluence and cell growth slows. The first two bars demonstrate that prostatic epithelial cells are depolarized when rapidly growing and hyperpolarize by about 20 mV when they reach confluence. The second pair of bars demonstrate that exponentially growing cells are depolarized even in growth factor deprived culture conditions (stripped serum) and hyperpolarize less in the absence of growth factors on reaching confluence. The final pair of bars demonstrate that cells grown in the presence of the active metabolite of testosterone, DHT (dihydroxytestosterone), are slightly hyperpolarized during exponential growth, but depolarize on reaching confluence.

These differences in cell membrane potential support the notion that growth conditions of prostatic epithelia in vivo will likely influence the cell membrane potential of prostatic epithelial cells. Cell membrane potential will influence the transepithelial potential measured at the prostate surface. Alteration in the DC potential measured trans-rectally in combination with impedance will be used to diagnose prostate cancer.

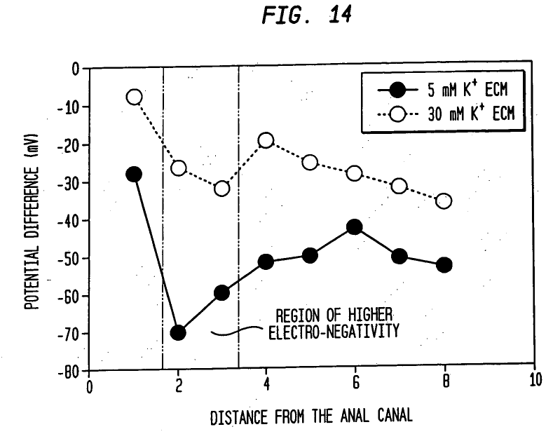

FIG. 14 demonstrates electropotential measurement made over the prostate of a patient with a normal prostate. The patient was undergoing a colonoscopy for screening, which was negative and had a normal PSA. The ECM (electroconductive medium) contained 5 mM K+ and physiological concentrations of other electrolytes. The filled circles and solid line represent the measurement of surface electropotential (y-axis) starting at 1 cm from the anal verge to 8 cm along the anterior aspect of the rectum (x-axis). The values increase from approximately −28 mV to −70 mV over the prostate and drop (depolarize) to approximately −52 mV over the top of the prostate, and referenced to the bloodstream. When the ECM is changed to a solution with the same osmolality, but with a K+ concentration of 30 mM. The electropotential of the surface of the rectal mucosa depolarizes to 30 mV (open circles joined by a dotted line). This indicates significant K+ permeability of the overlying rectal mucosa. The higher region of electro-negativity over the prostate is consistently seen when the prostate is healthy.

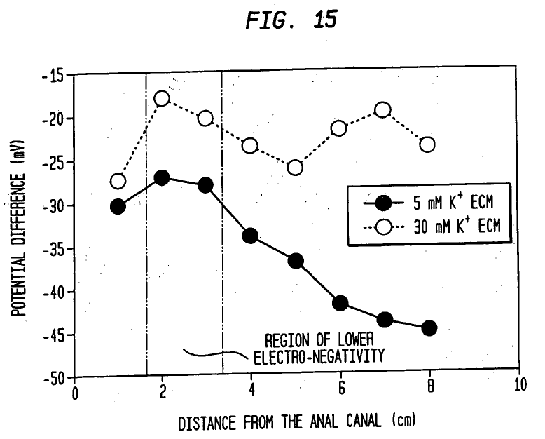

FIG. 15 demonstrates measurement made in a patient with a previously biopsied prostate cancer. The symbols and axes are the same as in FIG. 14. The region of electro-negativity is lower over the cancerous prostate. In this case electropotential measurements of between −26 and −27 mV were made over the cancerous lobe of the prostate i.e., 30 to 40 mV lower than observed over healthy prostate. When the ECM was changed to a solution with a K+-concentration of 30 mM a depolarization of 8-9 mV was observed, or about a third of that observed in healthy prostate. This indicates a decrease in K+-conductance and permeability of both the prostate and overlying rectal mucosa.

These changes in the normal DC electrical profile of the prostate will be used separately or in combination with AC impedance measurements to diagnose prostate cancer. Identification of depolarization of the prostate relative to the higher polarity of the surrounding rectal mucosa together with decreased K+-conductance indicate the presence of prostate cancer. Additional AC measurements with differently spaced electrodes will permit probing of the underlying prostate to accurately localize the site of the prostatic malignancy.

### EXAMPLE 5

### Chemopreventative and Therapeutic Use

In addition to the ionic, pharmacologic, and hormonal agents described above, the system and method of the present invention may be used with cancer preventative and therapeutic agents and treatments. Specifically, electrical measurement of altered structure and function provides a method for evaluating a patient's response to the drugs without requiring a biopsy and without waiting for the cancer to further develop. Patients who respond to a given chemopreventative or therapeutic agent would likely show restoration of epithelial function to a more normal state. Patients who do not respond would show minimal change or may even demonstrate progression to a more advanced stage of the disease. This system and method, thus, may be used by either clinicians or drug companies in assessing drug response or by clinicians in monitoring the progress of a patient's disease and treatment, or monitoring the process of carcinogenesis (cancer development), before an overt malignancy has fully developed.

Furthermore an understanding of the physiological basis of the altered impedance permits more accurate diagnosis. For example impedance may increase or decrease because of several factors. Increased stromal density of breast tissue may alter impedance. This is a non-specific change, which may not have any bearing on the probability of malignancy. On the other hand a decrease in potassium permeability of the epithelia around a developing malignancy would increase impedance and would be more likely associated with a developing cancer than a non-specific impedance change. Additional information is obtained from my method by probing the tissue to different depths using spaced voltage-sensing electrodes. The use of electrophysiological, pharmacological and hormonal manipulations to alter impedance differentially in normal compared to cancer-prone, pre-malignant or malignant tissue is another significant difference, which enhances the diagnostic accuracy of my invention over the above referenced one.

### EXAMPLE 6

### Electrophysiologic Approaches to Assessing Tumor Ablation Margins and Responses to Drug Therapy

Decisions with regard to how much tissue to resect at surgery or during endoscopy are usually based on the gross appearance of the lesion, or in the case of surgical resection on the tactile feel of the lump or tumor mass in relationship to the surrounding “normal” tissue. In surgical breast cases when the lesion is non-palpable, a wire localization may be performed, whereby the radiologist places a needle or wire under mammographic control and the surgeon resects the tissue around the wire. The specimen is x-rayed to ensure that the lesion has been removed. A pathologist may also examine the specimen and perform frozen sections to ensure that tumor does not infiltrate the margin. In which case additional tissue may have to be resected. The tumor may also be identified under ultrasound guidance for wire localization or intraoperatively to facilitate adequate resection margins. Such methods have limitations in that they do not always accurately determine the location of the tumor margins and thus in some cases cause tumor tissue to remain in the body, or in other cases, cause excess normal tissue to be removed.

As an improvement to these methods, electrophysiological measurements could be used preoperatively or intraoperatively to help identify the adequacy of resection. For example, a needle electrode could be placed into the tumor, and measurements made between the tumor and its surface and then the margin of the intended resection. In the case of breast cancer, impedance would be lower in the tumor compared to the surrounding tissue and a one cm margin of higher impedance surrounding tissue could be selected as an adequate “safe” margin for resection. Similarly DC potential measurements could be made by themselves or in combination with impedance whereby a positive deflection has been reported at the cancer-normal tissue interface, followed by a negative deflection within the center of the tumor.

Devices to measure or display the electrophysiological characteristics of tissue and the differences between normal and abnormal tissue may include those known in the art such as electrical meters, digital signal processors, volt meters, oscillators, signal processors, potentiometers, or any other device used to measure or display voltage, conductance, resistance or impedance.

DC potential is usually measured using a voltmeter, consisting of a galvanometer in series with a high resistance, and two electrodes (one working and one reference). Voltmeters may be analog or digital. Ideally these should have an extremely high input resistance to avoid current-draw. DC potential may also be measured with an oscilloscope.

Impedance may be measured using a number of approaches. Without limitation, examples include phase-lock amplifiers, which may be either digital or analog lock-in amplifiers. Pre-amplifiers may be used in conjunction with the lock-in amplifier to minimize stray currents to ground improving accuracy. Digital lock-in amplifiers are based on the multiplication of two sine waves, one being the signal carrying the amplitude-modulated information of interest, and the other being a reference signal with a specific frequency and phase. A signal generator can be used to produce the sine waves or composite signal to stimulate the tissue. Analog lock-in amplifiers contain a synchronous rectifier that includes a phase-sensitive detector (PSD) and a low-pass filter. Other approaches include the use of an impedance bridge with an oscillator to produce an AC sine wave. These devices when automated are referred to as LCR-meters and use an auto-balancing bridge technique. Constant current or constant voltage current sources may be used. In one preferred embodiment, a constant current source is used. Rather than an oscillator with a fixed frequency signal a signal generator, which produces, superimposed sine waves may be used.

In another preferred embodiment, the tissue response is deconvolved using fast Fourier transforms or other techniques. Bipolar, tripolar or tetrapolar current and voltage electrodes may be used to make measurements. In one preferred embodiment, tetrapolar electrode configurations are employed to avoid inaccuracies that are introduced due to electrode polarization and electrode-tissue impedance errors. Rather than impedance, current density may be measured using an array of electrodes at the epithelial or skin surface. Impedance may also be measured using electromagnetic induction without the need for electrode contact with the skin or epithelium.

In order to process large amounts of data, the methods of the present invention could be implemented by software on computer readable medium and executed by computerized equipment or central processor units. Readings from the devices disclosed herein can be displayed on various computer screens, monitors, or other displays known in the art.

Measurements could be made with multiple electrodes along a needle or through the use of separate needle electrodes consisting of current passing and voltage measuring electrodes. Alternatively needle electrodes could be used in combination with surface, nipple cup or ductal electrodes to identify “safe” margins where the electrophysiological characteristics revert to more “normal” parameters.

Pharmacological or hormonal agents could be used in conjunction with the above referenced approaches to characterize the tissue. For example, the electrophysiological response of the tissue to estradiol would be different when measured in normal tissue and compared with the response of malignant tissue. Such difference in response could be used in one embodiment of the present invention to detect such malignant tissue.

In the case of other hollow organs, such as bowel or stomach, surface electrodes on the serosal surface of the organ or introduced endoscopically could be used by themselves or in conjunction with needle electrodes to identify the “safe” margin for resection. In the case of endoscopic resection, these could be interfaced with an endoscope or used in a stand-alone fashion. Liver tumors could be assessed for adequate margins using a combination of needle and electrodes placed on the surface of the liver. Bile duct cannulation could be performed to make trans-biliary endothelial measurements similar to those described in the breast. Pancreatic resection margins could also be assessed using surface and transepithelial measurements after cannulating the pancreatic duct. Lung resections could also be performed using a bronchial electrode and surface electrodes to assess margins. These are just a few examples of this novel approach. Electrophysiological determination of “safe” margins for resection could be used in any other area of the body where a tumor or other abnormal tissue is sought to be removed.

In one preferred embodiment, a first electrode is inserted into a tumor sought to be removed and a second electrode is inserted into an adjacent area of tissue known to not contain tumor tissue. Electrophysiological characteristics of the two tissues are then taken. As noted above, the electrophysiological characteristic of the tumor tissue will be different from the electrophysiological characteristic of the normal tissue. The first electrode is then moved relative to its original location until the electrophysiological characteristic more closely matches the electrophysiological characteristic of the normal tissue. Surgical resection margins can be determined by locating the points at which these electrophysiological characteristics change.

In another preferred embodiment, only one electrode is used. A base-line electrophysiological measurements is taken of normal tissue and used to compare sequential measurements taken of the tissue sought to be resected.

In another preferred embodiment, only one electrode is used and the electrophysiological measurements of the selected area of tissue are compared to known values for normal tissue.

In a further embodiment, multiple electrodes are spaced along a single probe or other member, the member is inserted through the tumor or along the tumor and the electrophysiological measurements are taken from the multiple electrodes to develop a spatial mapping of signals over the distance of the probe.

In still further embodiments, multiple electrodes can be placed on a plate, sheet, web or flexible planar member that is placed over an area of tissue including an area to be removed, and electrophysiological map is created to determine the location of tissue with abnormal electrophysiological characteristics.

In still further embodiments, the electrodes can be placed on a glove to be used during examinations or surgical removal of tissue.

Ablative therapies include therapeutic approaches such as surgery, the use of physical agents such as photoablation (light), radiation, radiofrequency ablation, heat, laser treatment, or freezing (cryotherapy) to destroy tissues, or the administration of toxic agents such as ethanol or formaldehyde to a tumor to destroy it. A number of new approaches have been introduced as radiotherapy alternatives to treat breast cancer. These are collectively known as accelerated partial-breast irradiation (APBI), and include intraoperative radiation therapy, brachytherapy and 3-dimensional conformal radiotherapy. In all three of these approaches, as well as other treatments, it is necessary to know the extent of the tumor to plan treatment and then to monitor the patient for recurrence.

Novel approaches using biologics or agents for destroying inhibiting tumor growth, either directly, or by impacting the tumor microenvironment include new classes of cytotoxic drugs, agents or approaches that act via immune-stimulatory effects, agents that stimulate apoptosis, inhibit angiogenesis, or alter tumor cell signaling pathways, and agents targeted specifically to novel cancer cell targets. Physical approaches such as electroporation may be combined with new agents such as, genes, viral transfectants or drugs to obtain entry into tumor cells.

These physical or combination approaches may also be monitored electrophysiologically through the novel methods and devices disclosed herein. The effectiveness of alternative therapies for cancer treatment, including but not limited to herbal therapies, dietary supplements, bioactive food components, or unconventional pharmacological and biological interventions (e.g. antineoplastons, Coley's toxin, enzyme therapies, etc.) may also be evaluated using the embodiments described above. Similarly, drugs/agents, biologics, alternative therapies, radiation, heat, electroporation or surgery may be used as single agents/modalities or in combination for the treatment of early and advanced disease and these treatments may be monitored electrophysiologically with the methods and devices disclosed herein.

Electrophysiological measurements may be used to assess margins during and after ablative therapy or novel approaches using biologics or alternative therapies. In ablative treatments it is important to assess how much tissue needs to removed or destroyed at the time of treatment. Destruction of too much tissue may result in unnecessary damage to surrounding normal tissue. Inadequate tumor treatment so that microscopic cells are left at the margins of the tumor may result in subsequent recurrence of the tumor and treatment failure. Following ablative treatment or during treatments with drugs, novel approaches and alternative treatments monitoring of the response of the tumor to therapy is vital to identify which tumors are responding, progressing or recurring. This monitoring may be accomplished non-invasively using electrophysiological measurements of the present invention, saving costs, reducing patient discomfort, and increasing reliability and sensitivity of measurement over prior known approaches.

Another use of electrophysiological monitoring is during anatomical and molecular image guidance for targeted treatment with ablative techniques or delivery of chemotherapeutic agents. In these circumstances electrophysiological measurements may be used in combination with anatomic or molecular image guidance to more accurately localize the tumor for ablative techniques, or the delivery of chemotherapeutic or other biologic agents.

The embodiments described herein are described in reference to humans. However, cancers in non-humans may be also diagnosed with this approach and the present invention is also intended to have veterinary applications.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for determining the location of a tumor margin comprising:
measuring a first electrophysiological characteristic of an area of tissue to be removed;
measuring a second electrophysiological characteristic of an area of normal tissue; and
determining the location of a tumor margin based on the difference in the first electrophysiological characteristic of the area of tissue to be removed and the second electrophysiological characteristic of the area of normal tissue.

2. The method of claim 1, wherein the first electrophysiological characteristic and the second electrophysiological characteristic is an impedance of the tissue.

3. The method of claim 1, wherein the first electrophysiological characteristic and the second electrophysiological characteristic is the DC potential of the tissue.

4. The method of claim 1, further including introducing at least one agent into the area of tissue to be removed and the area of normal tissue.

5. The method of claim 1, wherein the area of normal tissue is adjacent to the area of tissue to be removed.

6. A method for determining the location of a tumor margin comprising:
measuring a first plurality of electrophysiological characteristics of an area of tissue to be removed at a first location;
measuring a second plurality of electrophysiological characteristics of an area of normal tissue at a second location; and
determining the location of a tumor margin based on the difference in first and second pluralities of electrophysiological characteristics.

7. The method of claim 6, wherein the first and second pluralities of electrophysiological characteristics are the impedance and DC potential of the tissue.

8. The method of claim 6, wherein the first and second plurality of electrophysiological characteristics are used to generate a electrophysiological profile of an area of tissue to be removed and the area of normal tissue.

9. The method of claim 6, further including introducing at least one agent into the area of tissue to be removed.

10. An apparatus for determining the location of a tumor margin comprising:
a first electrode for measuring a first electrophysiological characteristic of an area of tissue to be removed;
a second electrode for measuring a second electrophysiological characteristic of an area of normal tissue; and
a device operative to determine the difference between the first and second electrophysiological characteristics.

11. The apparatus of claim 10, wherein the area of normal tissue is adjacent to the area of tissue to be removed

12. The apparatus of claim 10, wherein the first and second electrodes are on the same member.

13. An apparatus for determining an area of tissue to be removed from a body comprising:
a first electrode for measuring a first electrophysiological characteristic of an area of tissue to be removed;
a second electrode for measuring a second electrophysiological characteristic of an area of normal tissue;
a device operative to display the first and second electrophysiological characteristics.

14. The apparatus of claim 13, wherein the first and second electrodes form portions of a member.

15. The apparatus of claim 13, wherein the first and second electrodes are on an endoscope.

16. The apparatus of claim 13, wherein the first and second electrodes are on a biopsy device.

17. The apparatus of claim 14, wherein the member is a flexible tube.

18. The apparatus of claim 14, wherein the member is a flexible substantially flat material.

19. An apparatus for determining an area of tissue to be removed comprising:
a first electrode for measuring a first electrophysiological characteristic of an area of tissue to be removed;
a second electrode for measuring a second electrophysiological characteristic of an area of normal tissue;
a first device operative to display the first electrophysiological characteristic; and
a second device operative to display the second electrophysiological characteristic.

20. The apparatus of claim 19, wherein the first device operative to display the first electrophysiological characteristic and the second device operative to display the second electrophysiological characteristic are in a single housing.

21. A computer-readable medium having computer-executable instructions for performing a method comprising:
measuring an electrophysiological characteristic of an area of tissue to be removed from an area of normal tissue;
determining the location of a resection margin between the area of tissue to be removed and the area or normal tissue based on the difference in the electrophysiological characteristic of the area of tissue to be removed from known values of the electrophysiological characteristic of normal tissue.

22. A computer-readable medium having computer-executable instructions for performing a method comprising:
measuring a first electrophysiological characteristic of an area of tissue to be removed;
measuring a second electrophysiological characteristic of an area of normal tissue; and
determining the location a margin between the area of tissue to be removed and the area or normal tissue based on the difference in the first electrophysiological characteristic of the area of tissue to be removed and the second electrophysiological characteristic area of normal tissue.

23. The computer-readable medium of claim 22, wherein the first electrophysiological characteristic and the second electrophysiological characteristic is the impedance of the tissue.

24. The computer-readable medium of claim 22, wherein the first electrophysiological characteristic and the second electrophysiological characteristic is the DC potential of the tissue.

25. The computer-readable medium of claim 22, further comprising introducing at least one agent into the area of tissue to be removed and the area of normal tissue.

26. A method for determining the resection margin of an area of tissue comprising:
measuring a plurality of electrophysiological characteristics of an area of tissue to be removed from an area of normal tissue; and
determining the location of a resection margin between the area of tissue to be removed and the area or normal tissue based on the difference in the electrophysiological characteristics of the area of tissue to be removed and known electrophysiological characteristics of normal tissue.

27. The method of claim 26, wherein the plurality of electrophysiological characteristics are impedance and DC potential.

28. The method of claim 26, wherein the plurality of electrophysiological characteristics are measurements of impedance taken from at least two locations within the area of tissue to be removed from an area of normal tissue.

29. The method of claim 26, wherein the plurality of electrophysiological characteristics are used to produce an electrophysiological profile of the area of tissue to be removed from an area of normal tissue.

30. The method of claim 29, wherein the electrophysiological profile is displayed on a monitor.

31. A method for facilitating the removal of an area of tissue comprising:
measuring the electrophysiological characteristic of the area of tissue to be removed;
comparing the electrophysiological characteristic of the area of tissue to be removed to the electrophysiological characteristic of an area of normal tissue; and
determining the location of the area of tissue to be removed based on the comparison between the first electrophysiological characteristic of the area of tissue to be removed and the electrophysiological characteristic of an area of normal tissue.

32. A method for determining the efficacy of a form of medical treatment comprising:
measuring a first electrophysiological characteristic of an area of tissue to be treated;
applying a treatment to the area of tissue to be treated;
measuring a second electrophysiological characteristic of the area of tissue to be treated;
comparing the first and second electrophysiological characteristics; and
determining the efficacy of the medical treatment based on the comparison between the first and second electrophysiological characteristics.

33. The method of claim 32, wherein the form of treatment is selected from the group of treatments consisting of ablation therapy, radiation therapy, cryotherapy, and drug therapy.

34. A method for determining the efficacy of a form of medical treatment comprising:
applying a treatment to an area of tissue;
measuring an electrophysiological characteristic of the area of tissue;
comparing the electrophysiological characteristic of the area of tissue to predetermined values;
determining the efficacy of the medical treatment based on the comparison between the electrophysiological characteristic and predetermined values.

35. A method for determining the efficacy of a form of medical treatment comprising:
applying a treatment to the area of tissue to be treated;
measuring a first electrophysiological characteristic of the area of treated tissue;
measuring a second electrophysiological characteristic of a second area of tissue;
comparing the first and second electrophysiological characteristics; and
determining the efficacy of the medical treatment based on the comparison between the first and second electrophysiological characteristics.

36. The method of claim 35, wherein the second area of tissue is normal tissue.

37. The method of claim 35, wherein the second area of tissue is adjacent to the area of tissue to be treated tissue.

38. The method of claim 35, further comprising introducing an agent into the area of tissue to be treated.

39. An apparatus for determining the efficacy of a form of treatment comprising:
a first electrode for measuring a first electrophysiological characteristic of an area of tissue to be treated;
a second electrode for measuring a second electrophysiological characteristic of an area of normal tissue;
a first device operative to display the first electrophysiological characteristic; and
a second device operative to display the second electrophysiological characteristic.

40. An apparatus for determining the efficacy of a form of treatment comprising:
a first electrode for measuring a first electrophysiological characteristic of an area of tissue to be treated;
a second electrode for measuring a second electrophysiological- characteristic of an area of normal tissue;
a device operative to determine the difference between the first and second electrophysiological characteristics.

41. A computer-readable medium having computer-executable instructions for performing a method for determining the efficacy of a form of treatment comprising:
applying a treatment to an area of tissue;
measuring an electrophysiological characteristic of the area of tissue;
comparing the electrophysiological characteristic of the area of tissue to an electrophysiological characteristic of normal tissue; and
determining the efficacy of the medical treatment based on the comparison between the electrophysiological characteristic of the area of tissue and the electrophysiological characteristic of normal tissue.
